(19) United States
(12) Patent Application Publication
(10) Pub. No.: US 2024/0074465 A1
(43) Pub. Date: Mar. 7, 2024

(54) FOOD IRRADIATION DOSE UNIFORMITY

(71) Applicant: Starfire Industries LLC, Champaign, IL (US)

(72) Inventors: Robert A. Stubbers, Savoy, IL (US); Brian E. Jurczyk, Champaign, IL (US); Thomas J. Houlahan, JR., Urbana, IL (US); Matthew D. Coventry, Champaign, IL (US); Darren A. Alman, Mahomet, IL (US)

(21) Appl. No.: 18/242,499

(22) Filed: Sep. 5, 2023

Related U.S. Application Data
(60) Provisional application No. 63/403,405, filed on Sep. 2, 2022.

(51) Int. Cl. A23L 3/26

(52) U.S. Cl. CPC A23L 3/263

(57) ABSTRACT

A food irradiation system including a plurality of compact linac systems is described herein. Each compact linac system, of the plurality of compact linac systems, includes: a high energy particle beam source providing a particle beam at up to 10 MeV; an emission target assembly configured to generate bremsstrahlung x-rays when impacted by particles of the particle beam; and a drift tube through which the particle beam passes on a path from the high energy particle beam source to the emission target assembly. The emission target assembly is positioned at a distal end of the drift tube for direct impingement of the particle beam to generate the bremsstrahlung x-rays in a directed radiation beam. Ones of the plurality of compact linac systems are individually positioned such that, as a group, the plurality of compact linac systems provide directed radiation beam coverage at prescribed radiation dose levels for an overall cumulative volume.

### CROSS-REFERENCE TO RELATED APPLICATIONS

This application is a non-provisional of U.S. Provisional Patent Application No. 63/403,405, filed on Sep. 2, 2022, entitled “FOOD IRRADIATION DOSE UNIFORMITY,” the contents of which are hereby incorporated by reference in their entirety.

### TECHNICAL FIELD

The present disclosure generally relates to treatment of food to eliminate biological pathogens. More particularly, the disclosure relates to use of high-energy to perform such treatments.

### BACKGROUND OF THE INVENTION

The present disclosure addresses several needs for systems and associated methods of operation of such systems for carrying out high-energy e-beam and x-ray phytosanitary treatment and food irradiation to eliminate pests, invasive species, fungus, mold, and other hazards to shore up global food security and increase shelf life. Present-day phytosanitary and irradiation methods primarily include hot water soak (destroy texture, food properties), cold treatment (flash freeze), methyl bromide exposure (fumigation), Co-60 gamma-ray irradiation (radioactive source), and bremsstrahlung x-ray irradiation from a low-energy anode (x-ray tube) or high-energy target (e-beam converter in electron accelerator). In addition, high-energy x-rays and electrons can be used to replace other methods for material sterilization methods (such as chemical ethylene oxide treatment) for bulk materials, medical devices, skin care products, fabrics, additives, bio-organic materials and feedstocks, seeds, etc.

X-ray and gamma-ray are preferable treatment means that address/overcome drawbacks of chemical treatments that have lasting carcinogenic pathways. However, current technology based on 3-5 MCi radioactive Co-60 is a major security/diversion risk with limited global supply. Treatment using electron bremsstrahlung x-ray generation with conventional electrostatic systems has been limited to 150 kV to 350 kV range with x-ray tube/window converters. The former being implemented using conventional x-ray tube technology used in the medical and inspection industries. 1-5 MeV-class electrostatic systems, such as the Pelletron or Tandetron, allow lower-frequency DC-like efficiency at a cost of physical size and weight using transmission converters, such as W or Ta. These systems are typically limited in total power and not suitable for high-power irradiation. High-current electromagnetic/RF accelerators operate in the 1-10 MeV range with transmission-type x-ray converters (up to 7.5 MeV) and transmission e-beam windows (up to 10 MeV). The latter systems are typically services with extremely large recirculating electron devices (e.g. Rhodotrons) or S-Band linacs devices. Because of the large size of systems needed for electron generation and acceleration, a single beam line is typically used to transport the e-beam into a magnetic deflection sweeper (horn) that spreads electron heating over a large area and angle to generate x-rays, or through a thin window to allow the electron beam to raster over the object to be irradiated directly. To reach economies of scale, large-scale electron accelerators require 250-1000 kW to generate 40-240 kW of power onto transmission Ta bremsstrahlung convertors or thin-foil windows. The large devices operate quasi continuously for irradiating pallets or large shipments of goods in a single-sided fashion due to the large magnetic steering horn and >40 kW power levels.

Irradiation of food or materials are often subject to a minimum dose requirement to achieve sprout inhibition, insect disinfestation, shelf-life extension, pathogen reduction and elimination of pests. Similarly, the food or materials are often subject to a maximum dose limitation for quality, taste, structure, etc. Thus, pallets or boxes receiving radiation will have a resultant radiation dose profile over the volume that is challenging for single-sided irradiation. The challenges of achieving sufficient doses of radiation using single-sided irradiation are mitigated by running pallets or boxes of material through the scanning system 2 to 4 times with the material rotated 90 or 180 degrees relative to the large accelerator on subsequent passes. This process decreases the throughput of a physical plant (¼×) and increases the physical cost and footprint (4×) for conveyance and material transport. Improvements in the physical size, power handling and complexity of the irradiation system could reduce this negative.

Energy conversion efficiency is important for devices operating continuously or high duty over a year from an OpEx perspective. Electrostatic acceleration offers >75% AC-to-DC, CW operation and large acceptance for electron beam generation. Electromagnetic (RF) power is less efficient <<50% AC-to-RF resulting in higher operating cost in addition to higher electronics cost that is about $3-10/W vs. $1-2/W higher cost relative to DC $/W. Electromagnetic (MW) is even less efficient. However, electromagnetic power supplies offer the advantage of a decrease in system size. Traditional electrostatic accelerators become massive in size and scaling above a few MeV accelerating energy make those systems costly as well. Traditional RF/MW accelerators are very high power (>40 kW) to achieve economies of scale for irradiation. Innovations in both electrostatic, laser-based and RF/MW accelerators potentially support/enable new ways for irradiation, and thus address drawbacks of current systems described herein above.

The system and method of operation of the present disclosure addresses the problems of known systems, leverages advances in high-voltage/high-gradient materials, electron accelerator dynamics, and provides efficient power coupling/delivery. An x-ray converter design facilitates providing new approaches for improved IAEA-accepted 5 MeV bremsstrahlung photon sources for pallet irradiation of food for phytosanitary and pathogen reduction. Systems according to the present disclosure provide an up to 7.5 MeV x-ray source and up to 10 MeV electron beam direct irradiation system.

### SUMMARY OF THE INVENTION

In accordance with the present disclosure, a food irradiation system including a plurality of compact linac systems is described herein. Each compact linac system, of the plurality of compact linac systems, includes: a high energy particle beam source providing a particle beam at up to 10 MeV; an emission target assembly configured to generate bremsstrahlung x-rays when impacted by particles of the particle beam; and a drift tube through which the particle beam passes on a path from the high energy particle beam source to the emission target assembly. The emission target assembly is positioned at a distal end of the drift tube for direct impingement of the particle beam to generate the bremsstrahlung x-rays in a directed radiation beam. Ones of the plurality of compact linac systems are individually positioned such that, as a group, the plurality of compact linac systems provide directed radiation beam coverage at prescribed radiation dose levels for an overall cumulative volume.

Additionally, a pallet irradiation system is described that includes the above-summarized food irradiation system and a conveyance apparatus configured to carry a pallet supporting an object to be irradiated.

### BRIEF DESCRIPTION OF THE DRAWINGS

While the appended claims set forth the features of the present disclosure with particularity, the present disclosure, together with its objects and advantages, may be best understood from the following detailed description taken in conjunction with the accompanying drawings of which:

FIG. 1 is a prior art graphic commonly used to represent the positive effects of cold pasteurization and food irradiation for fruits, vegetables, meats, grains and spices.

FIG. 2 is a photo and schematic of a prior art Co-60 irradiator facility with batch processing conveyance, where the photo is of Co-60 tubes in the storage pool emitting Cherenkov light.

FIG. 3A is a prior art graphic rendering of two 40-80 kW S-Band microwave linacs in a single-sided firing configuration to irradiate one or two pallets traveling transversely.

FIG. 3B is a prior art graphic rendering showing more of the irradiation processing facility with conveyance, chicanes, and turnstiles for 90 deg and 180 deg rotation of material for dose control in a thick shielded bunker room.

FIG. 4 is a prior art graphic rendering showing a 160-240 kW high-power recirculating RF accelerator in a side firing configuration with a large magnetic steering horn on a single converter target with material conveyance in a very thick shielded bunker room.

FIG. 5 is a prior art graphic rendering depicting a system to improve dose uniformity in a single-sided firing configuration by a stop-and-scan process rotating individual pallet stacks 360 degrees.

FIG. 6A is a prior art graphical illustration depicting the energy-angle correlation and spatial distribution for x-ray bremsstrahlung spectra as a function of incident electron energy onto a thin target.

FIG. 6B is a prior art graphical illustration of the relative x-ray intensity from a tungsten 0.8 mm thick target for a given electron energy including forward and backward x-ray percentages.

FIG. 6C is a prior art graphical illustration of the x-ray intensity as a function of angle for a set of different target converter materials showing the Z dependence on total yield, angular spread forward/backward radiation distribution.

FIG. 6D is a prior art graphical illustration of the total x-ray conversion efficiency separated into forward radiation and backward radiation for a fixed 5 MeV electron beam onto different thicknesses of W target.

FIG. 6E is a prior art graphical illustration of the total x-ray conversion efficiency separated into forward radiation and backward radiation for a fixed Ta target thickness with variable electron energies, showing effect on x-ray spectral distribution.

FIG. 7A is a prior art graphical illustration depicting the x-ray dose profile from a 5 MeV electron beam onto a 0.8 mm Ta converter into a reference water volume showing the 10× asymmetry from single-sided irradiation and reduction to 2× from double-sided irradiation.

FIG. 7B is a prior art graphical summary of FIG. 7A along the dose centerline to highlight the dual-sided improvement in dose superposition in dose uniformity.

FIG. 8A is a prior art schematic depicting the generic state-of-the-art electron utilization scheme where the e-beam is generated in a particle accelerator, the electrons pass into a beamline to a beam steering horn to either a converter assembly (for x-rays) or a thin-window for electron emission into air.

FIG. 8B is a prior art photograph of an S-band linac for e-beam emission on a crane gantry to lower the system through the roof of a facility due to size, weight and vertical mounting over a conveyance.

FIG. 8C is a prior art photograph of a electron beam scanning horn showing the coils and cores for the electron beam scanning system, tight focused electron rastering pattern over the thin extraction foils and air-jet cooling for e-beam irradiation.

FIG. 8D is a prior art illustration of the emission target region at the end of the scanning horn with Ta converter, direct water cooling and stainless-steel backer.

FIG. 9A illustratively depicts an emission target assembly to emit electron radiation for the present disclosure highlighting basic structural elements for coupling with an electron beam accelerator for a compact linac system, including a vacuum-facing boundary, optional thermal conductor, cooling layer and optional end capping layer to atmosphere in accordance with the present disclosure.

FIG. 9B illustratively depicts the emission target assembly to emit x-ray radiation for the present disclosure highlighting basic structural elements for coupling with an electron beam accelerator for a compact linac system, including a vacuum-facing capping layer, converter material, anti-corrosion interface layer, cooling layer, and end capping layer to atmosphere in accordance with the present disclosure.

FIG. 9C further illustrates an emission target assembly to emit x-ray radiation utilizing a liquid converter target material that can also serve as the direct cooling structure through flow to an external heat sink; also shown is additional low-energy x-ray attenuation for beam hardening in accordance with the present disclosure.

FIG. 9D illustratively depicts geometric shaping of converter target material within the emission target assembly to present an angled surface for an incident electron beam to modify effective thickness for on-axis bremsstrahlung generation vs. angle to modify bremsstrahlung x-ray energy-angle spectrum anisotropy in accordance with the present disclosure.

FIG. 10A further illustrates another aspect of the present disclosure to modify bremsstrahlung x-ray energy-angle spectrum anisotropy by engineering a crystal structure orientation or bulk structure orientation of a converter material to be substantially orthotropic with an electron beam in accordance with the present disclosure.

FIG. 10B further illustrates another aspect of the present disclosure to modify the bremsstrahlung x-ray energy-angle spectrum anisotropy by engineering the crystal structure orientation or bulk structure orientation of the converter material to be substantially orthogonal with the electron beam in accordance with the present disclosure.

FIG. 11A and FIG. 11B schematically depict a compact linac system comprising an electron accelerator directly coupled to an extended-snout drift tube utilizing beam spread to illuminate an emission target assembly to generate an energy-angle distribution of bremsstrahlung photons for different accelerating energies of 5 MeV and 3 MeV respectively in accordance with the present disclosure.

FIG. 12A and FIG. 12B schematically depict a shield collimator closely-coupled to an extended-snout drift tube relatively positioned to an emission target assembly to affect bremsstrahlung radiation energy-angle distribution to control forward photons and equivalent dose profile in accordance with the present disclosure.

FIG. 12C and FIG. 12D schematically depict a shield collimator closely coupled to an extended-snout drift tube to physically collimate and constrain forward photons into a symmetric shaped beam or an asymmetric shaped beam for irradiating objects and obtaining certain dose profiles in accordance with the present disclosure.

FIG. 12E schematically depicts a shield collimator with castellations to promote the trapping of backward radiation to minimize scattering to redirect into forward radiation with lower effective energy in accordance with the present disclosure.

FIG. 13 schematically depicts a compact linac system comprising an electron accelerator directly coupled to an extended-snout drift tube utilizing beam spread to illuminate an emission target assembly to generate a forward-directed electron beam for different accelerating energies in accordance with the present disclosure.

FIG. 14A schematically depicts a compact linac system comprising an electron accelerator directly coupled to an extended-snout drift tube incorporating a beam deflector capable of moving the electron beam to illuminate an emission target assembly in an expanded beam region to generate a rastered electron beam for different accelerating energies in accordance with the present disclosure.

FIG. 14B schematically depicts a compact linac system comprising an electron accelerator directly coupled to an extended-snout drift tube incorporating a beam deflector capable of spreading the electron beam to illuminate an emission target assembly in an expanded beam region to generate multiple and/or overlapping electron beams for different accelerating energies in accordance with the present disclosure.

FIG. 15A schematically depicts an ultra-compact irradiation apparatus suitable for phytosanitary and sterilization treatment comprising one or more compact linac systems arrayed about an object conveyance for in-line exposure in accordance with the present disclosure.

FIG. 15B schematically depicts variable placement of compact linac systems azimuthally around an in-line object conveyance apparatus for high-throughput phytosanitary and sterilization treatment in accordance with the present disclosure.

FIG. 15C schematically depicts an ultra-compact irradiation apparatus suitable for phytosanitary and sterilization treatment such as the one depicted in FIG. 15A wherein one or more compact linac systems are pivoted longitudinal about an object conveyance for in-line exposure in accordance with the present disclosure.

FIG. 16 schematically depicts an ultra-compact irradiation apparatus suitable for phytosanitary and sterilization treatment in FIG. 15A-C wherein one or more compact linac systems are stacked vertically alongside an object conveyance apparatus for in-line exposure to irradiate larger objects, such as stacked pallets in accordance with the present disclosure.

FIG. 17A and FIG. 17B schematically depicts dynamic energy-angle control using relative position shield collimators to adjust directed radiation beam towards an object to be treated to adjust the relative dose profile across the object in accordance with the present disclosure.

FIG. 18A and FIG. 18B schematically depicts a central feature of the present disclosure regarding placement of multiple—four in this example—compact linac systems in close proximity to an object positioned such that multiple directed radiation beams with overlapping radiation fields will levelize a normalized dose across the object to improve the dose uniformity ratio and decrease spread between Dₘₐₓ and Dₘᵢₙ in accordance with the present disclosure.

FIG. 19A and FIG. 19B further extends the schema of FIG. 18A-B by adding additional compact linac systems positioned around the object to be irradiated to further levelize normalized dose across an object to improve dose uniformity ratio, increase throughput and treatment speed; wherein individual adjustment of each compact linac system “source”, adjustment of collimation for energy-angle correlations, adjustment of acceleration energy, adjustment of low-energy filters for beam hardening, adjustment of beam current (flux), adjustment of beam position, adjustment of exposure time, etc is supported in accordance with the present disclosure.

FIG. 20 schematically depicts an ultra-compact irradiation apparatus suitable for phytosanitary and sterilization treatment comprising many compact linac systems arrayed about an object conveyance for in-line exposure, e.g. 20-40, 4 kW units would project 80-160 kW beam power as a lower-cost, small facility size replacement for conventional large single-sided irradiation platforms in accordance with the present disclosure.

FIG. 21A and FIG. 21B schematically depicts the ultra-compact irradiation apparatus employing local radiation shielding with small-diameter access ports for compact linac systems with emission target assemblies and shield collimators greatly reducing backwards photons and stray radiation burden on facility, personnel and equipment to enable close placement of compact linac systems to each other for overlapping radiation fields, high area and volume density for dose uniformity and throughput in accordance with the present disclosure.

FIG. 22 schematically depicts another embodiment of the present disclosure using magnetic electron transport to guide and direct the electron beam to an emission target assembly located further away from the electron accelerator or at an angle to the accelerator to produce a directed radiation beam in accordance with the present disclosure.

FIG. 23A and FIG. 23B and FIG. 23C further illustrates FIG. 22 extended to multiple compact linac systems arranged to produce overlapping radiation fields from multiple directed radiation beams surrounding a region that could contain objects to be irradiated in accordance with the present disclosure.

FIG. 24 is a flowchart summarizing operations of a method to achieve improved dose uniformity ratio and improved linear exposure time for an ultra-compact irradiation apparatus in accordance with the present disclosure.

FIG. 25 is a flowchart summarizing operations of a method for using radiation detectors placed near an object to be irradiated in accordance with the present disclosure.

FIG. 26 is a detailed schematic illustration of the apparatus to achieve differential adjustment to radiation dosing and monitoring of the actual delivered dose conditions to the object under irradiation in accordance with the present disclosure.

FIG. 27 is a schematic illustration of the method of FIG. 25 and the apparatus of FIG. 26 for operational dose control (e.g. 400Gy±20Gy) and auditable verification for each object irradiated in accordance with the present disclosure.

### DETAILED DESCRIPTION OF ILLUSTRATIVE EXAMPLES

The description that follows, including a detailed of the figures, is not to be taken in a limiting sense but is made merely for the purpose of describing the principles of the described embodiments.

Food security is critical to our nation. For phytosanitary applications, such as fruit and vegetable treatments, there is a requirement for disinfestation and quarantine. Typically, fumigation is performed (why you wash your vegetables before eating). However, in mid-2022 methyl bromide was environmentally phased out causing a major disruption for the importation of certain fruits and vegetables. Co-60 irradiation is the natural solution. However, the Co-60 gamma source supply is constrained and the cost has increased from $1.8 to $6.4/Ci in 2022 and is expected to peak at $10/Ci in 2024 due to global supply and demand.

The present disclosure pertains to a system for an ultra-compact apparatus and irradiation method for phytosanitary and food security treatment with small size/power/cost that facilitates distributed point-of-packaging irradiation, add-on to existing cold-storage/container facilities, and is an enabler for small-scale medical sterilization, industrial non-destructive testing, and materials processing.

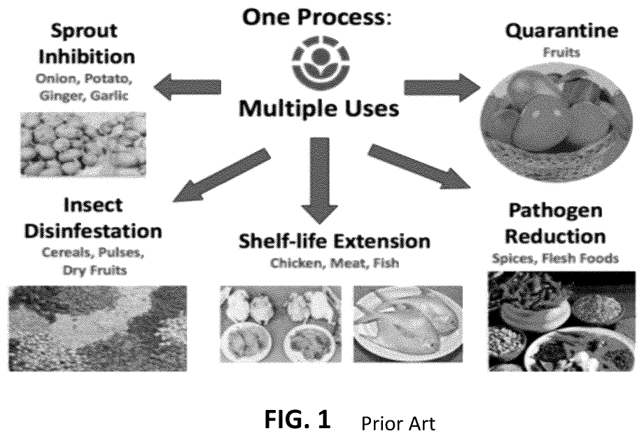

The present disclosure provides a pathway for small-enough systems that are able to fit through doors and existing radiation shields to enable retrofitting Co-60 gamma-ray facilities disrupted by global supply chain difficulties, as well as lower capital expenditure and operational expenditure costs to enable more end-users to adopt accelerator irradiation for its benefits shown in FIG. 1 which is a graphic commonly used to represent positive effects of cold pasteurization and food irradiation for fruits, vegetables, meats, grains, and spices.

Food has lower/upper bounds on irradiation dose set by the cognizant country agency, e.g. in the US it is the FDA, USDA, APHIS, etc. Dose Uniformity Ratio, the ratio of the highest over the lowest dose in the sample, yields a good metric for evaluating approaches. A high DUR also means more time spent dwelling on an object to achieve a minimum irradiated dose metric to satisfy a cognizant agency's metric. Too high DUR can exceed thresholds for food damage. More importantly, lower DUR typically results in higher facility throughput, reduced energy costs and higher Gross Margin for an irradiation facility.

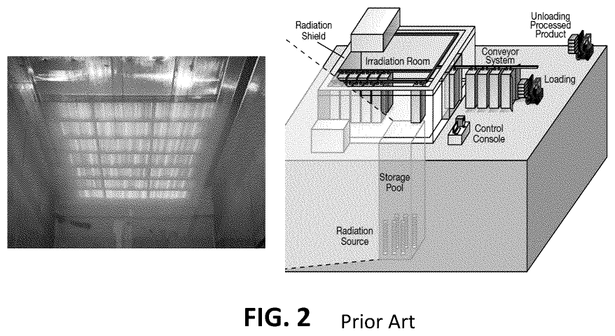

FIG. 2 is a graphic illustration of a Co-60 irradiator facility with batch processing conveyance and a photograph of Co-60 tubes in a storage pool emitting Cherenkov light. Co-60 systems are typically operated in a batch processing mode where objects (products) are loaded into transportable racks and moved into a shielded irradiation room. Radioactive Co-60 material is raised out of a storage pool into the middle of the room and generous MeV gamma-rays irradiate the materials. Co-60 systems typically route pallets through a room with exposure to 1, 2 or 4 sides of the pallet to improve dose uniformity across the package. DUR values of 2.0-2.5 are common for Co-60 systems. For Co-60, the gamma-ray energy is nearly monoenergetic (2 close photons) resulting in a particular depth vs. dose profile for a given effective density on the pallet. For more-dense materials, this results in higher dose near the outer edge vs. the middle of the pallet because of the depth profile. This shortcoming is exacerbated by single side irradiation and improved with 2-sided and 4-sided irradiation. Rotating the pallets 90 degrees four times allows for passage through an irradiation zone to achieve dosing. In 2023, the cost of Co-60 increased by over a factor of 4 on the global market and is constrained by limited supply with trade restrictions.

There are two primary MeV accelerator options: the 40-80 kV S-Band microwave electron linear accelerator (linac) and the 120-240 kW RF recirculating electron accelerator (Rhodotron). The Rhodotron technology operates at 400 kHz thereby minimizing switching losses and skin depth effects for higher efficiency—almost 2× better than conventional S-band linacs at GHz frequencies; however, this comes at a cost of size, minimum power and upfront capital expenditure. Large electron accelerators in the >>10 kW class typically raster the electron beam using bending/steering magnets to spread the thermal load over a large area—and this is done in conjunction with a horn-like converter target so produce a fan shaped beam. The magnetic steering system and large-area format target is costly, large in size, and heavy requiring cranes to install. In addition the thermal management systems are a significant fraction of the overall system. This emission target assembly can either comprise an x-ray converter to take the electrons and convert into bremsstrahlung radiation or comprise a thin vacuum window to enable direct electron beam irradiation treatment. The latter has the advantage of direct electron-to-electron energy transfer for higher efficiency vs. the former electron to photon to electron energy transfer with the reduced solid angle for total energy transfer efficiency. The advantage of x-ray is that the photons can penetrate deep into the object before generating a Compton electron for local energy transfer into dose; whereas, electrons (being charged) are immediately slowed down and attenuated near the surface of the object resulting in exaggerated dose profiles and very high DUR ratings.

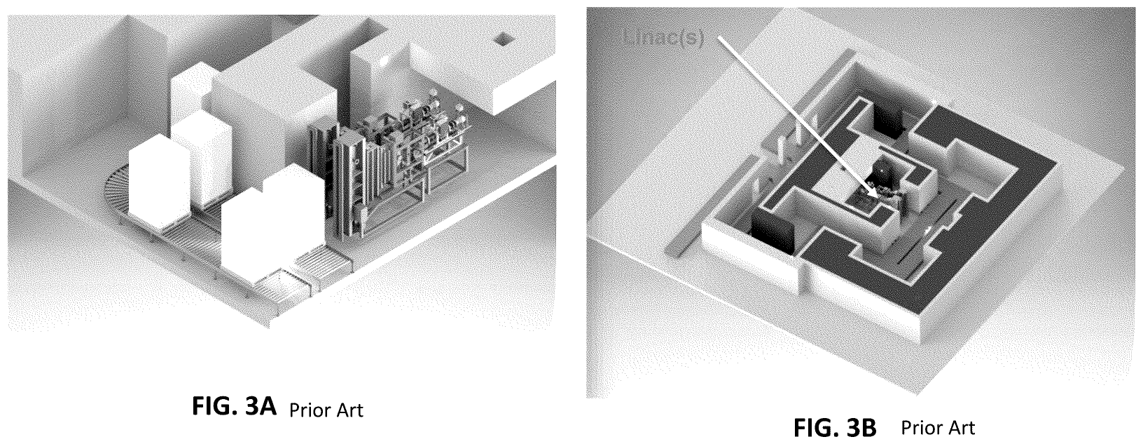

FIG. 3A is a graphic rendering of two 40-80 kW S-B and microwave linacs in a single-sided firing configuration to irradiate one or two pallets traveling transversely across the large magnetic scanning horn. Conventional techniques for full-pallet (40″×48″×96″) irradiation from single point electron beam source (40-240 kW into e-beam) use scanning horns to spread thermal and dose loading, leading to lost dose/inefficiency in pallet/object corners. This results in overdosing certain regions and underdosing others. For food safety, all of the object must receive a minimum dose and not exceed a maximum dose. This puts constraints on objects leading to systems with pallet rotation and wide-fan beams leading to wasted radiation, cost and expense. FIG. 3B is a prior art graphic rendering showing more of the irradiation processing facility with conveyance, chicanes, and turnstiles for 90 deg and 180 deg rotation of material for dose control in a thick shielded bunker room. A pallet of material will run through the scanning system 2 to 4 times with the material rotated 90 or 180 degrees relative to the large accelerator on subsequent passes. This process decreases the throughput of a physical plant (¼×) and increases the physical cost and footprint (4×) for conveyance and material transport. This can be improved from a system level approach in accordance with the present disclosure as described later.

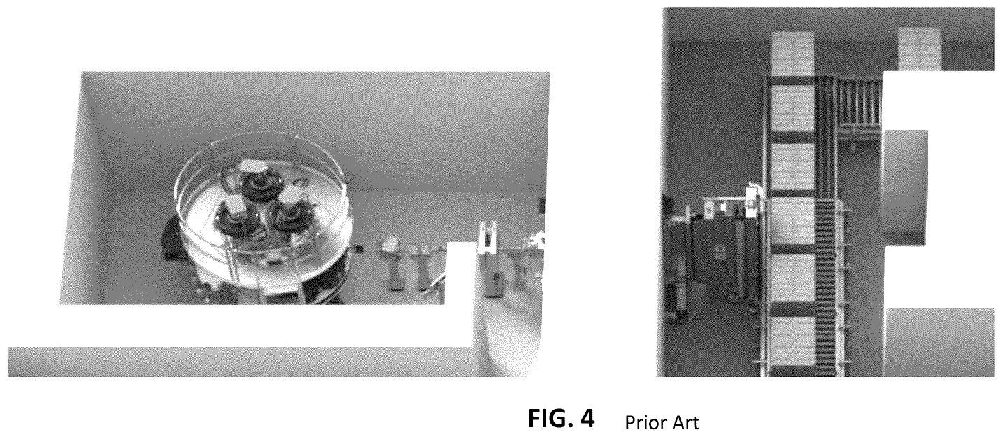

FIG. 4 is a prior art graphic rendering showing a 160-240 kW high-power recirculating RF accelerator in a side firing configuration with a large magnetic steering horn on a single x-ray converter target with material conveyance in a very thick shielded bunker room. For 5 or 7.5 MeV Ta x-ray bremsstrahlung accelerator systems using a single beamline with magnetic deflector onto a horn target producing a fan beam, the higher peak energy of the photons leads to greater penetration and Compton scattering within the center of the pallet improving the DUR compared to Co-60. 5 MeV x-ray systems typically achieve DUR values from 1.8-3.5. 7.5 MeV x-ray systems can achieve DUR values of 1.4-2.5. Lowest values use single station irradiation with rotating pallets for 360-degree spot illumination.

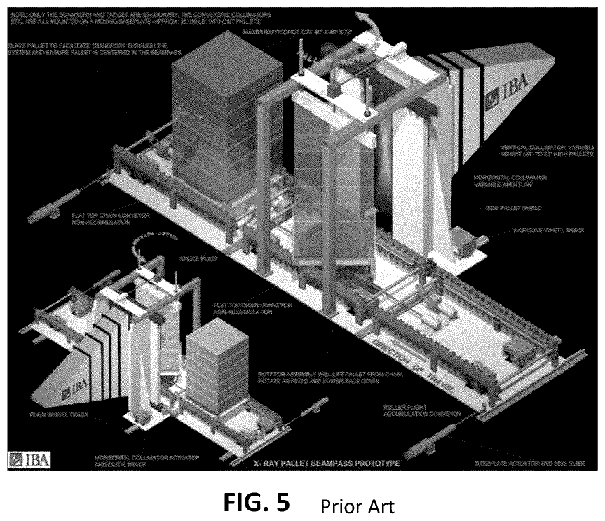

FIG. 5 is a graphic rendering depicting a system to improve dose uniformity in a single-sided firing configuration by a stop-and-scan process rotating individual pallet stacks 360 degrees. These systems have reduced throughput from mechanical staging and suffer from beam loss on the edges (i.e. top, bottom, sides) with a single fan beam sweeping from a large magnetic steering horn.

For direct e-beam irradiation, the primary driver is very high electrical efficiency into dose. However, for large pallets and dense objects, the e-beam penetration depths are shallow with steep dose profiles, which leads to large DUR values and overexposure on the edges and underexposure in the middle.

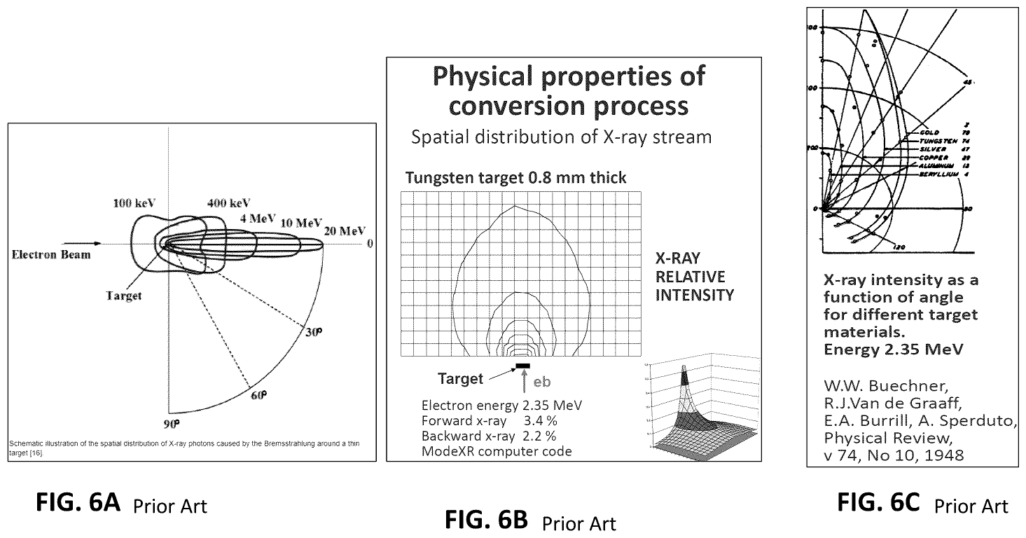

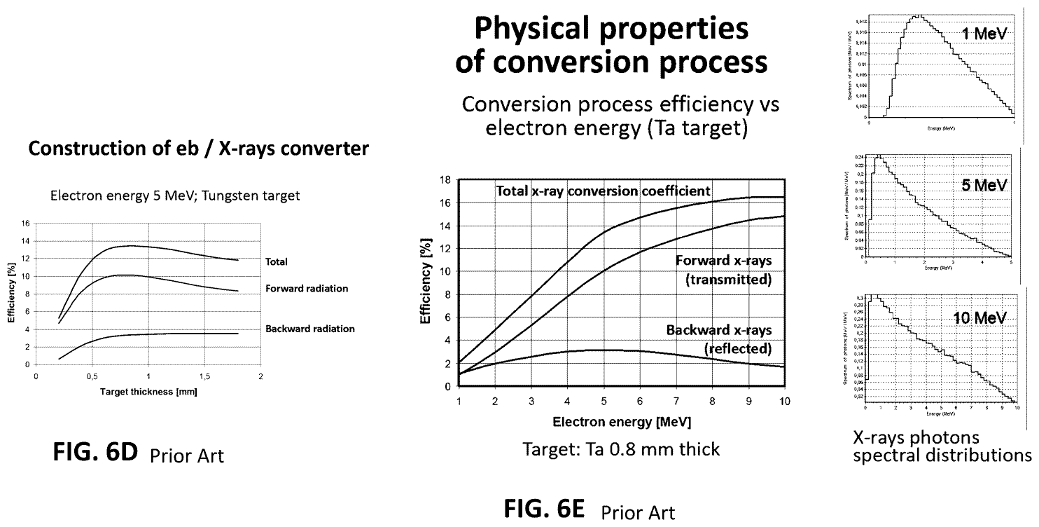

For x-ray irradiation, it is important to understand the energy-angle correlation and conversion from e-beam into photons. FIG. 6A illustratively depicts an energy-angle correlation and spatial distribution for x-ray bremsstrahlung spectra as a function of incident electron energy onto a thin target. FIG. 6B graphically summarizes relative x-ray intensity from a tungsten 0.8 mm thick target for a given electron energy including forward and backward x-ray percentages. FIG. 6C graphically depicts x-ray intensity as a function of angle for a set of different target converter materials showing the Z dependence on total yield, angular spread forward/backward radiation distribution. FIG. 6D graphically depicts total x-ray conversion efficiency separated into forward radiation and backward radiation for a fixed 5 MeV electron beam onto different thicknesses of W target. FIG. 6E graphically depicts total x-ray conversion efficiency separated into forward radiation and backward radiation for a fixed Ta target thickness with variable electron energies, illustratively showing effect on x-ray spectral distribution.

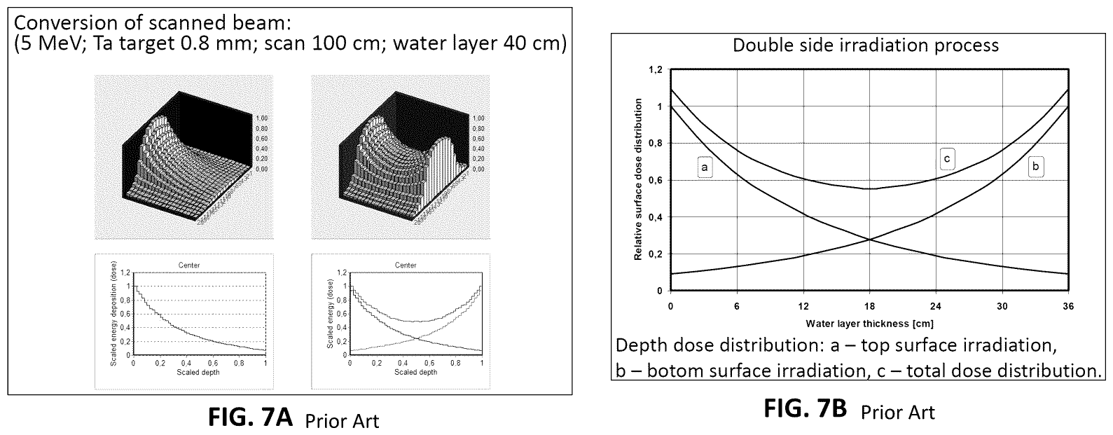

To understand how spatial and energy-angle distribution of the x-rays can be used, FIG. 7A graphically depicts x-ray dose profile from a 5 MeV electron beam onto a 0.8 mm Ta converter into a reference water volume showing a 10× asymmetry from single-sided irradiation and a reduction to a 2× difference in received dose achieved using double-sided irradiation. FIG. 7B graphically summarizes the results achieved by two-sided irradiation in accordance with FIG. 7A along the dose centerline to highlight the dual-sided improvement in dose superposition in dose uniformity. This can be improved from a system level approach in accordance with the present disclosure provided herein below.

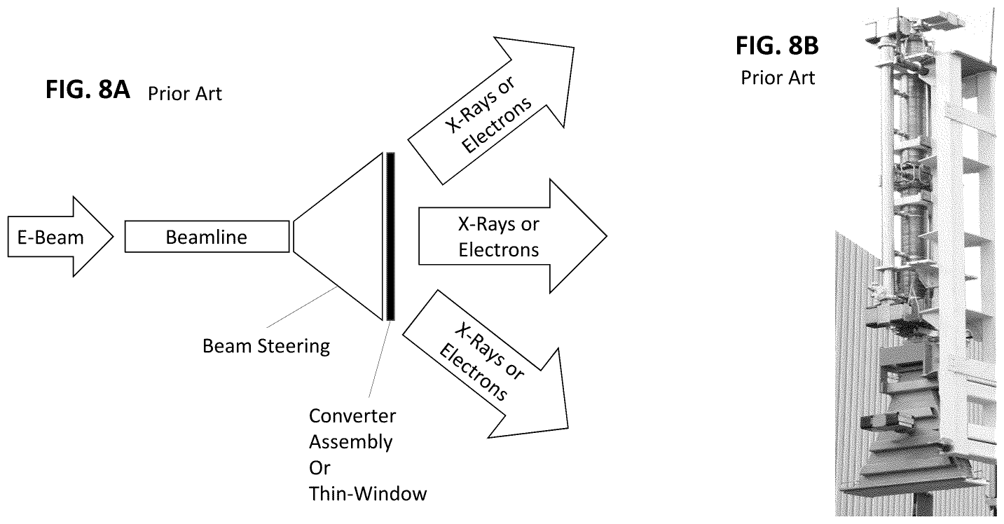

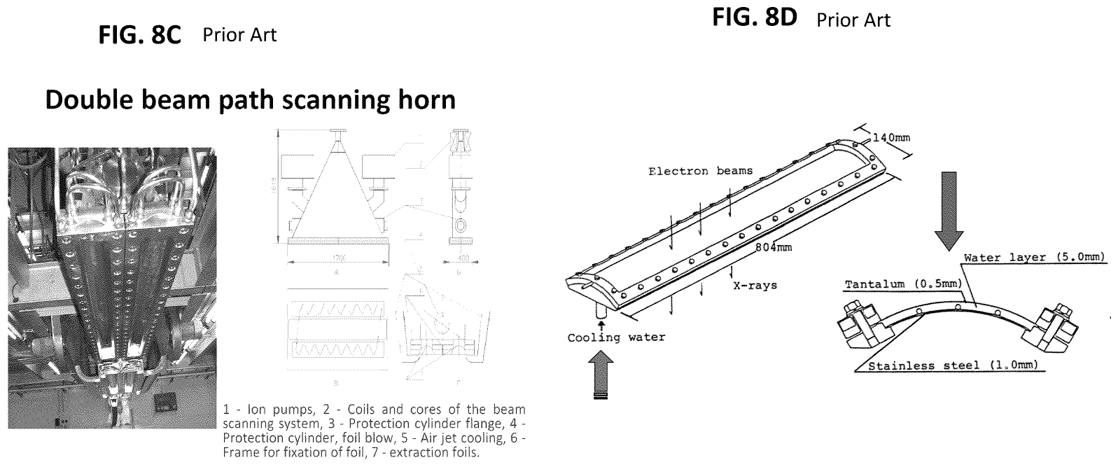

From a systems-level approach, there are many challenges with a 40-240 kW electron beam incident onto an emission target. FIG. 8A is a prior art schematic depicting a generic state-of-the-art electron utilization scheme where an e-beam is generated in a particle accelerator, and the electrons pass into a beamline to a beam steering horn to either a converter assembly (for x-rays) or a thin-window for electron emission into air.

FIG. 8B is a photograph of a known S-band linac for e-beam emission on a crane gantry to lower the system through a roof of a facility due to size, weight and vertical mounting over a conveyance apparatus.

FIG. 8C is a photograph of a known electron beam scanning horn for e-beam irradiation, including coils and cores for an electron beam scanning system providing a tightly focused electron rastering pattern over thin extraction foils and an air-jet cooling apparatus.

FIG. 8D illustratively depicts an emission target region at an end of a scanning horn with Ta converter, direct water cooling and stainless-steel backer.

Electron-to-X-ray bremsstrahlung conversion efficiency at 5 MeV is approximately 10% for W and 13% at 7.5 MeV for Ta high-temp targets into the forward 2 pi steradian direction. The usable fraction of the converted forward photons is approximately ½ this value at about 6% and 8% respectively. This is due to the predominance of low-energy x-rays below 1 MeV that are unable to penetrate deeply into palletized objects and contribute to shallow dose—skewing the DUR and are filtered out—and a large number of photons emitted at solid angles greater than 1 sr that don't make it to the object. Increasing electron energy improves the forward-directedness of the photons, shifts more of the spectrum to >1 MeV and improves penetration into pallet objects. Use of tungsten and uranium above 5 MeV results in neutron emission from electron-nuclear photodisintegration. Tantalum and bismuth are acceptable up to 7.5 MeV before onset of significant neutron emission. The IAEA limit in electron energy is 5 MeV due to the neutron effect and only in select countries has the limitation been increased to 7.5 MeV for Ta targets. The higher-energy results in a ‘harder’ spectrum of x-ray generation that is also more forward directed. The higher energy photons can penetrate deeper onto materials suitable for irradiating pallets of materials or higher density objects. The more forward directed the x-rays are, the greater the potential is for utilizing the radiation conversion for object irradiation—such as pallets. For the lower-energy x-rays, the radiation distribution is more spherical with additional loss from photons traveling in a wrong direction. Improvements to conventional converter targets can increase overall system level efficiency.

High-gradient DC accelerator technologies and high-gradient X-band microwave accelerator technologies enable kW-class (1-10 kW) MeV acceleration of electrons (5-10 MeV) with 1 m×0.5 m×0.5 m form factor length scales with lower capital expenditure and operational expenditure costs. As a result, there are options for an ultra-compact irradiation apparatus including multiple kW-class compact linacs that generate electron beams or x-rays photon beams with novel converter sources for irradiation, and provide multiplexed radiation beams around an object for improved DUR while maintaining high facility throughput with a single-pass in-line system. Furthermore, having a distributed set of radiation beams and associated corresponding radiation detectors provides real-time volumetric dose feedback with opportunity for adjustment.

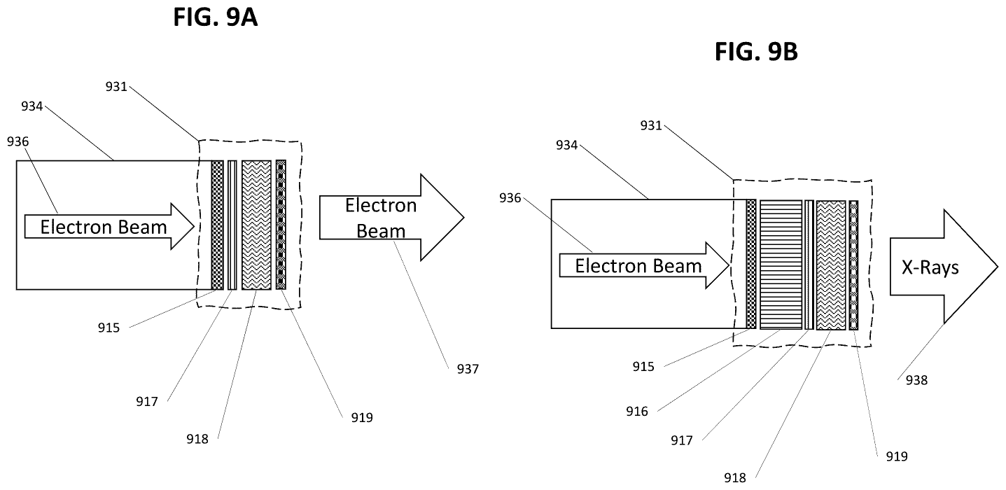

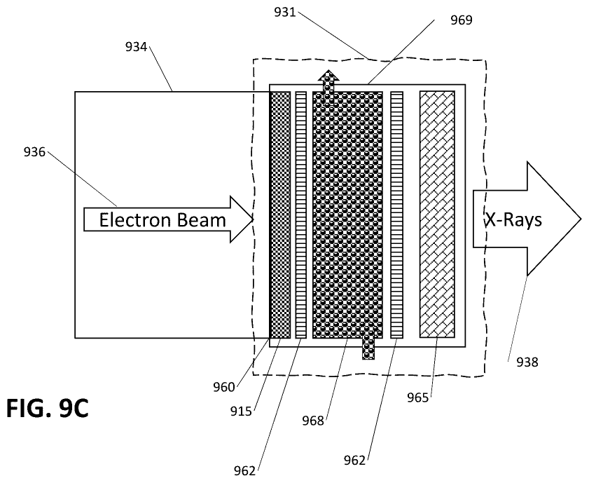

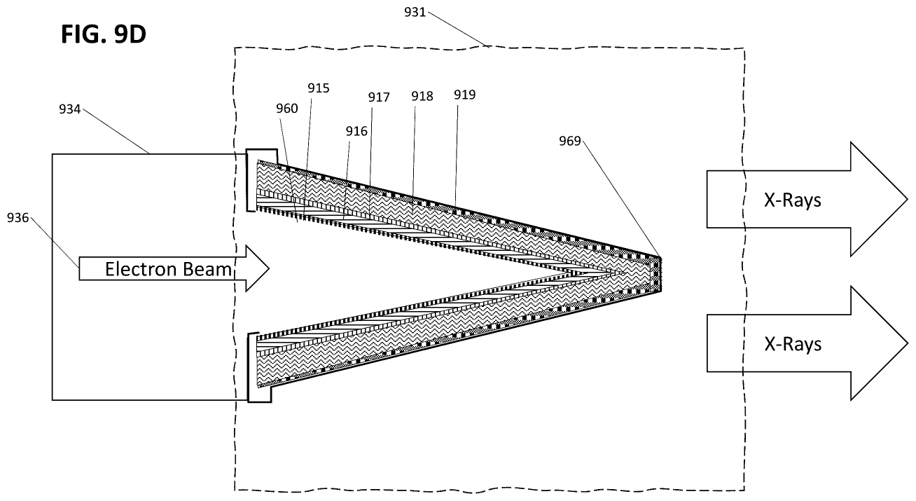

Turning attention first to an emission target assembly, a 1-10 kW class electron accelerator source does not need the very large and complicated magnetic scanning horns shown in the prior art systems discussed herein above because the power density is much lower and the emission target can be physically smaller, on the order of 0.1-10 cm diameter for its active region utilizing high-heat flux cooling solutions and phase change materials. The small active region size minimizes atmospheric pressure force across the vacuum boundary allowing thinner materials compared to the prior art at 40-400× as large. FIG. 9A illustratively depicts in accordance with the present disclosure, an emission target assembly 931 configured to emit radiation in the form of a forward electron beam 937 highlighting the basic structural elements for coupling with an electron beam trajectory 936 from an accelerator (not shown to the left) through a drift tube 934 for a compact linac system, including a vacuum-facing boundary 915, optional thermal conductor 917, optional cooling layer 918 and optional end capping layer 919 to atmosphere. Here a vacuum-facing boundary is a thin foil with low vapor pressure, high thermal tolerance, and sufficient structural strength to withhold atmospheric pressure on one side and hard vacuum on the other, e.g. Al, Ti, Be or alloy, brazed into the assembly and joined to the vacuum drift tube 934. The vacuum-facing boundary 915 can be coated with a thin capping layer facing the vacuum size to resist oxidation, corrosion, contamination or other weakening of the vacuum seal.

FIG. 9B illustratively depicts the emission target assembly 931 configured to emit x-ray radiation 938 in accordance with the present disclosure highlighting the basic structural elements for coupling with the electron beam trajectory 936 from the accelerator (not shown to left) through the drift tube 934 for a compact linac system, including the vacuum-facing boundary 915, the converter material 916, optional thermal conductor 917, cooling layer 918, and end capping layer 919 to atmosphere. Again the vacuum-facing boundary 915 can be coated with a thin capping layer facing the vacuum side to resist oxidation, corrosion, contamination or other weakening of the vacuum seal.

FIG. 9C further illustratively depicts an emission target assembly 931 configured to emit x-ray radiation 938 utilizing a liquid converter target material 968 that can also serve as the direct cooling structure through flow to an external heat sink (not shown). Also shown is an additional low-energy x-ray attenuation for beam hardening 965 structure that can be placed at the distal end after an anti-corrosion interface layer 962. A high-melting point, low-vapor pressure material for a capping layer 960 sits atop the vacuum-facing boundary 915. Surrounding this assembly is an emission target assembly enclosure 969 configured to provide hermeticity and flow paths for the liquid converter target material 968. The liquid can be a pure metal, such as Hg, or a low-temperature alloy (such as Bi, Pb, In, Sn eutectic) with margin for phase change expansion, or a heterogeneous liquid with high-Z material. In the case of room-temperature liquid Hg, the photofission threshold increases above 5.5 MeV. Therefore, similarly to U, the target material is suitable for the IAEA standard of 5 MeV. However, Bi is like Ta and can increase to 7.5 MeV without issues. The advantage of a liquid converter is a reduction in need for a separate cooling layer that adds additional matter in the path of the x-rays and the object.

FIG. 9D illustratively depicts geometric shaping of the converter target material 916 within the emission target assembly 931 configured to present an angled surface for the incident electron beam 936 to modify the effective thickness for on-axis bremsstrahlung generation vs. angle to modify the bremsstrahlung x-ray energy-angle spectrum anisotropy. The illustratively depicted configuration affects side lobe photon flux and has implications for reduced radiation shielding. Here the entire assembly is shaped into a V or a cone-like structure and is encapsulated in an assembly enclosure 969, including the vacuum facing boundary layer 915, with capping layer 960, optional thermal layer 917, cooling layer 918, and end capping layer 919. The geometric shape of the illustrative example spreads thermal loading from an electron beam over a larger surface area for greater thermal conduction and convection extraction, as well as presents two different path lengths for electrons generating photons and scattering.

Note that the thermal layer and cooling layer can comprise a thermal spreader, heat pipe, vapor chamber, liquid cooling, or heat transfer material. Also, a low-energy attenuator can be used to scatter/minimize low-energy photons from reacting objects to lower the shallow surface radiation dose. The low-energy attenuator serves to harden the spectrum and can be integrated with the assembly enclosure 969 or it can be external to the compact linac system and placed downstream in the directed radiation beam.

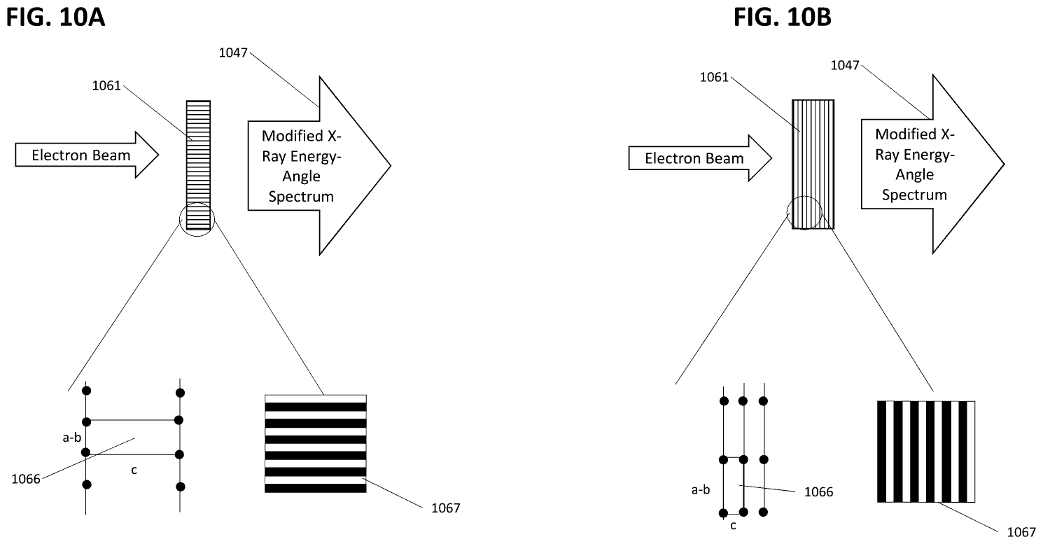

The selection of the converter material's crystal structure at the atomic lattice parameter, as well as the macroscopic grain orientation or layering can have an appreciable impact on energy-angle spectrum for the emitted radiation. FIG. 10A further illustrates another aspect of the present disclosure regarding modifying bremsstrahlung x-ray energy-angle distribution 1047 and spectrum anisotropy by engineering crystal structure orientation 1066 or bulk structure orientation 1067 of a converter material 1061 to be substantially orthotropic with the electron beam. FIG. 10B further illustrates another aspect of the present disclosure to modify the bremsstrahlung x-ray energy-angle distribution 1047 and spectrum anisotropy by engineering the crystal structure orientation 1066 or bulk structure orientation 1067 of the converter material 1061 to be substantially orthogonal with the electron beam. These two mutually exclusive atomic arrangements influence channeling of the emitted bremsstrahlung radiation to promote forward-directedness or to channel low-energy photons in the x-y plane vs. the electron beam z-axis. The former is good for increasing the effective electrical conversion efficiency of e-beam to photons and the latter is better for minimizing the lower-energy shallow dose to the object that impacts DUR.

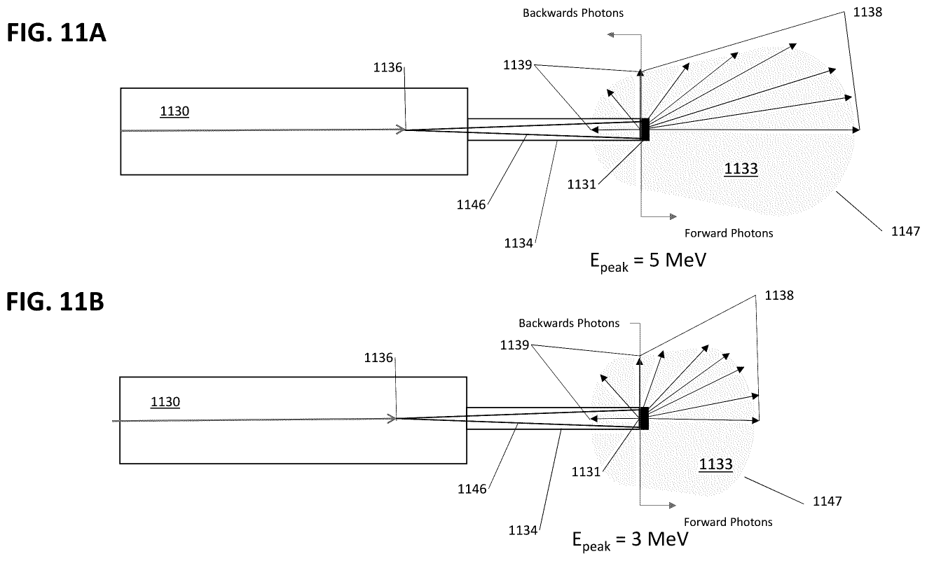

FIG. 11A and FIG. 11B schematically depict examples of a compact linac system comprising an electron accelerator 1130 directly coupled to an extended-snout drift tube 1134 injecting an electron trajectory 1136 to undergo beam spread 1146 to illuminate the emission target assembly 1131 and to generate an energy-angle distribution 1147 of bremsstrahlung forward photons 1138 and backward photons 1139 for different accelerating energies of 5 MeV and 3 MeV respectively. Emitted radiation 1133 encompasses energy-angle distribution 1147 shown by arrows and grey areal extent.

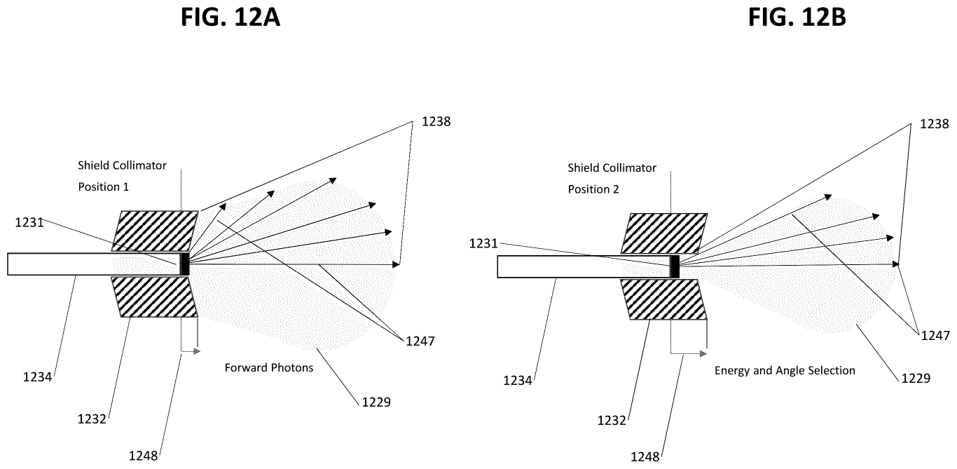

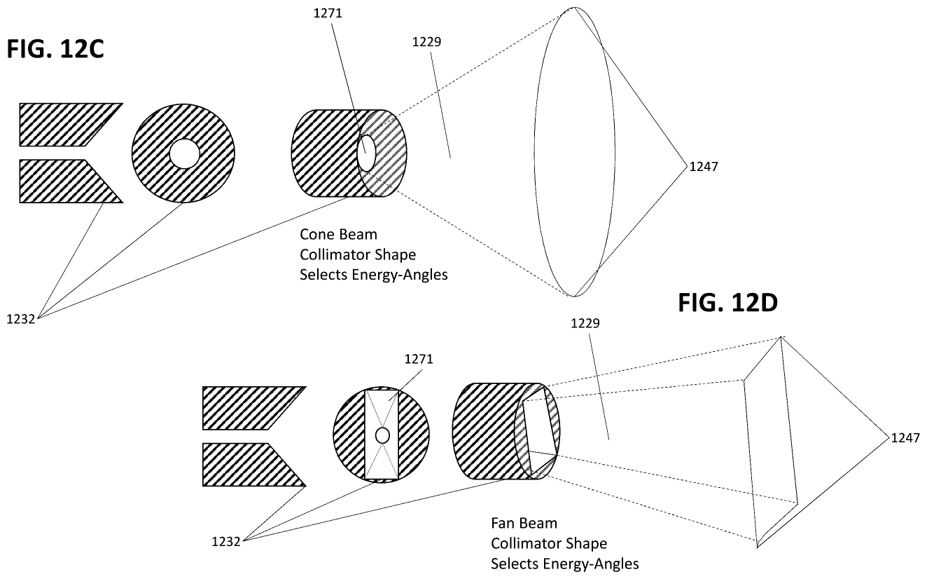

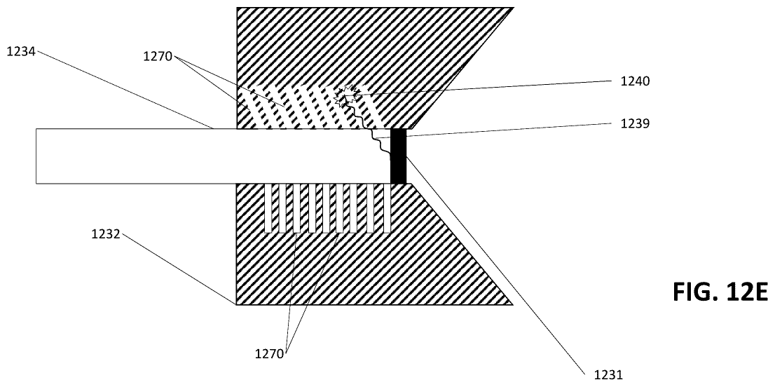

The compact linac system could be (for example) an X-band linac using split cavity high-gradient acceleration cells or a high-gradient DC linac with a sealed vacuum tube to achieve MeV acceleration on a tabletop. Close coupling enables a small-diameter emission target assembly suited for close coupling with a shield collimator for minimal size and weight compared to the present state-of-the-art magnetic steering horn and associated shielding. FIG. 12A and FIG. 12B schematically depict a shield collimator 1232 closely-coupled to an extended-snout drift tube 1234 relatively positioned (as indicated by arrow 1248) to the emission target assembly 1231 to affect an bremsstrahlung radiation energy-angle distribution 1247 to control forward photons 1238 and a resulting directed radiation beam 1229. This adjustment of the shield collimator 1232 and relative positioning (per arrow 1248), enables adjustment of the equivalent dose profile downstream in the object by modifying the resulting directed radiation beam 1229.

Furthermore, style of shield collimator used is variable depending on a desired set of directed radiation beam spatial characteristics. FIG. 12C and FIG. 12D schematically depict a shield collimator 1232 closely coupled to the extended-snout drift tube (not shown) to physically collimate and constrain the forward photons in the resulting directed radiation beam 1229 into a symmetric shaped beam or an asymmetric shaped beam for irradiating objects and obtaining certain dose profiles depending on a collimator exit shape 1271. Typically, a cone beam is used for simplicity and machining cost since the shield collimator is typically made from tungsten for high-Z and density. Additional physical structures/features to mitigate side lobe and scattered radiation can be found in FIG. 12E that is a schematic depiction of the shield collimator 1232 with castellations 1270 to promote trapping of backward radiation 1239 to minimize scattering 1240 to redirect into forward radiation with lower effective energy.

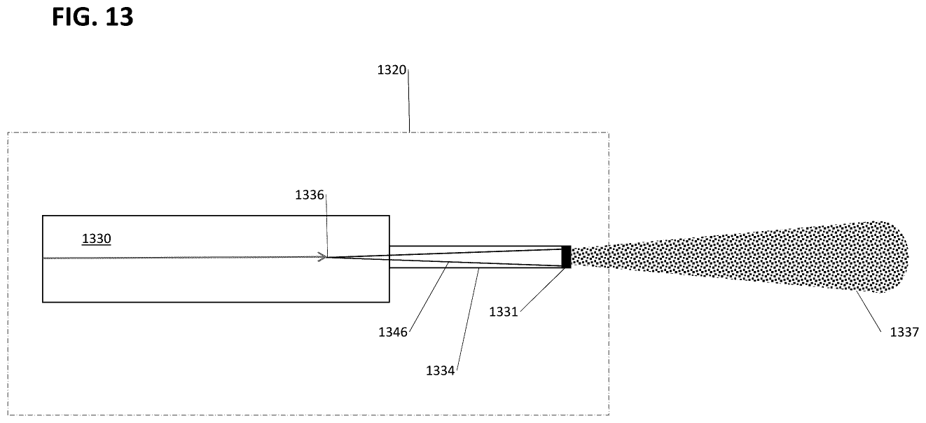

FIG. 13 schematically depicts a compact linac system 1320 comprising an electron accelerator 1330 directly coupled to an extended-snout drift tube 1334 utilizing beam spread 1346 from an electron trajectory 1336 to illuminate an emission target assembly 1331 to generate a forward electron beam 1337 for different accelerating energies. Note spatial distribution of the forward electron beam 1337 is fixed based on electron beam impact on a target within the emission target assembly 1331. This limited field of view can be mitigated by multiplexing multiple compact linac systems 1320 together into a patchwork line or quasi continuous area for irradiation.

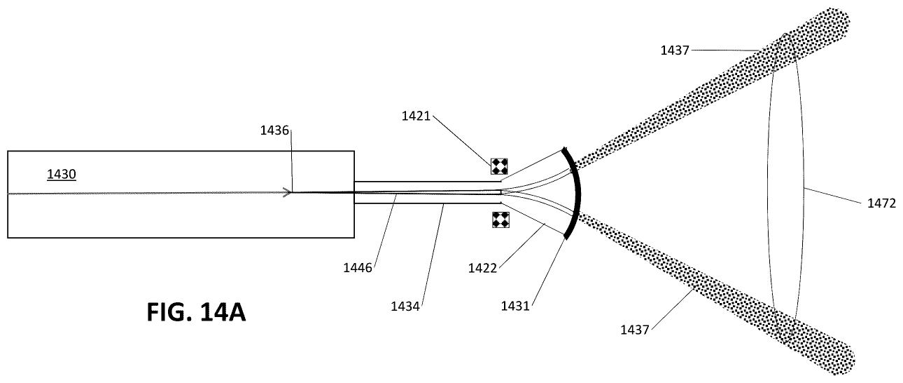

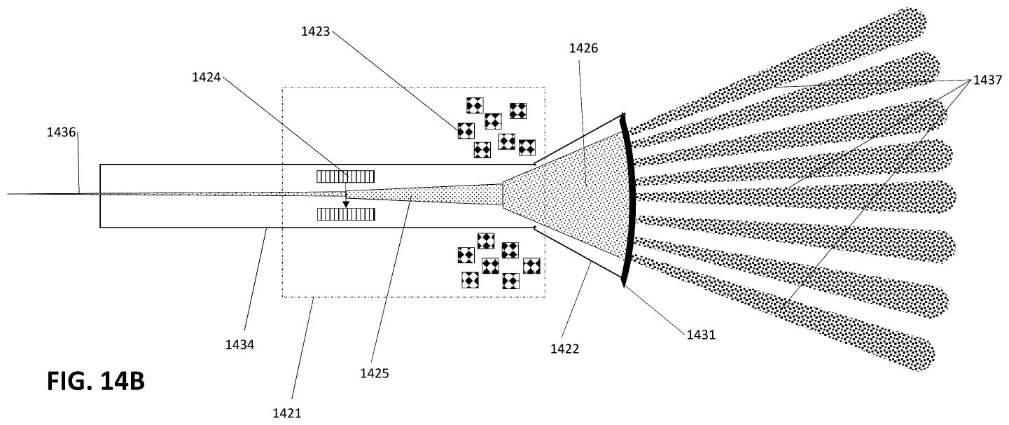

A hybrid approach is shown in FIG. 14A. A schematic depiction of a compact linac system 1420 comprising an electron accelerator 1430 directly coupled to an extended-snout drift tube 1434 incorporating a beam deflector 1421 capable of moving electron beam trajectory 1436 with a beam spread 1446 to illuminate an emission target assembly 1431 in an expanded beam region 1422 to generate a forward electron beam 1437 that is rastered over a rastered beam region 1472 for different accelerating energies. Note the principle of this approach is similar to the above-summarized/discussed magnetic scanning horns having a programmed rastering pattern to create an effective forward electron beam distribution.

FIG. 14B schematically depicts a compact linac system 1420 comprising an electron accelerator 1430 directly coupled to an extended-snout drift tube 1434 incorporating a beam deflector 1421 capable of spreading the electron beam trajectory 1436 to illuminate an emission target assembly 1431 in an expanded beam region 1422 to generate simultaneous multiple and/or overlapping forward electron beams 1437 for different accelerating energies. The beam deflector 1421 can further be expanded into x-y steering electrodes 1424 spreading the electron beam trajectory 1436 into an x-y electron beam 1425 that passes into a disordered magnetic array 1423 to transform into an r-O electron beam that broadly illuminates the emission target assembly 1431. The net result from this embodiment is the forward electron beam reaches a much wider and broader region for irradiation in quasi steady state.

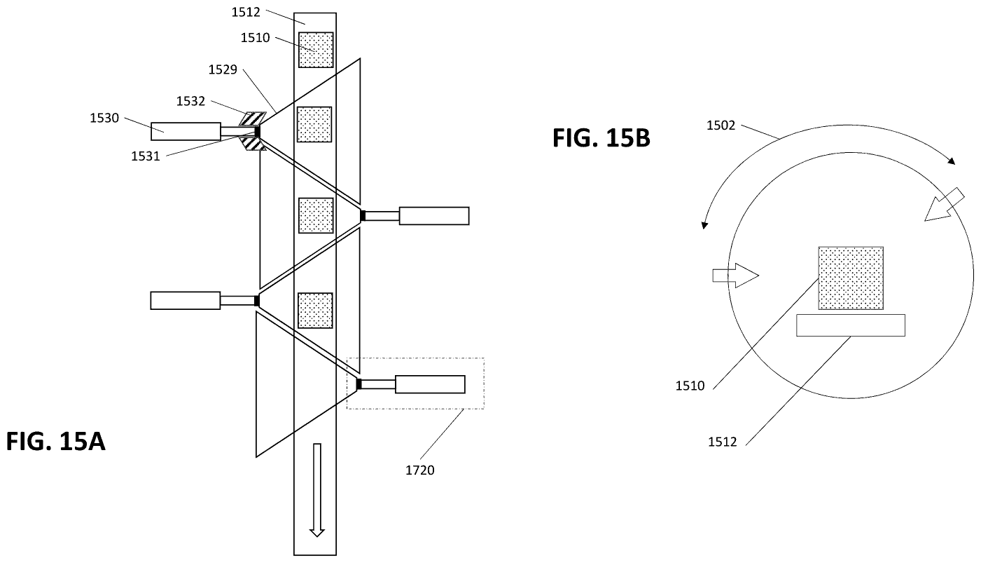

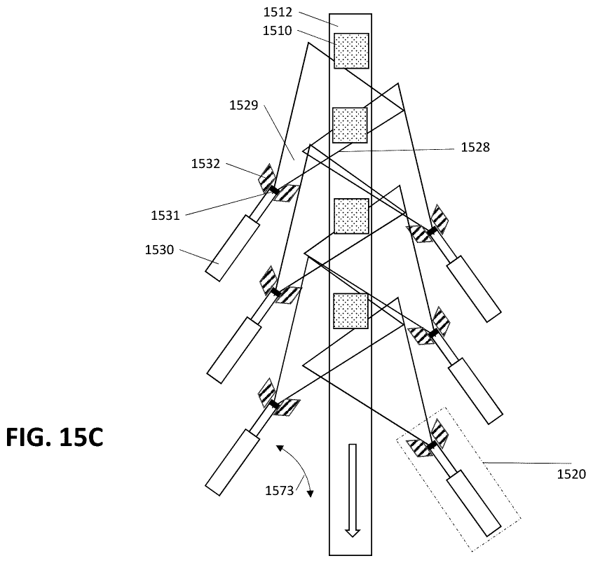

Having described several examples of emission sources, attention is now directed to system level architectures/assemblies for carrying out irradiation that comprise an assembly of multiple integrated compact linac systems with material conveyance. FIG. 15A schematically depicts an ultra-compact irradiation apparatus 1501 suitable for phytosanitary and sterilization treatment comprising one or more compact linac systems 1520 arrayed about an object 1510 conveyance 1512 apparatus for in-line exposure of objects. The compact linac systems 1520 contain the electron accelerator, emission target assembly, shield collimators, and produce a directed radiation beam 1529 that illuminates an object 1510 moving along a conveyance 1512 apparatus.

Multiple compact linac systems provide radiation dose from multiple angles. FIG. 15B illustratively depicts azimuthal placement 1502 of compact linac systems 1520 around the in-line object 1510 conveyance 1512 apparatus for high-throughput phytosanitary and sterilization treatment.

FIG. 15C schematically depicts the ultra-compact irradiation apparatus 1501 in a configuration suitable for phytosanitary and sterilization treatment in FIG. 15A wherein the one or more compact linac systems 1520 are pivoted into a longitudinal orientation 1573 about the object 1510 conveyance 1512 apparatus for in-line exposure. Multiple directed radiation beams 1529 align along the conveyance 1512 apparatus producing overlapping radiation fields 1528. In accordance with the illustrative example, the compact linacs 1530 can be freely oriented along the conveyance 1512 for dose control on the object 1510.

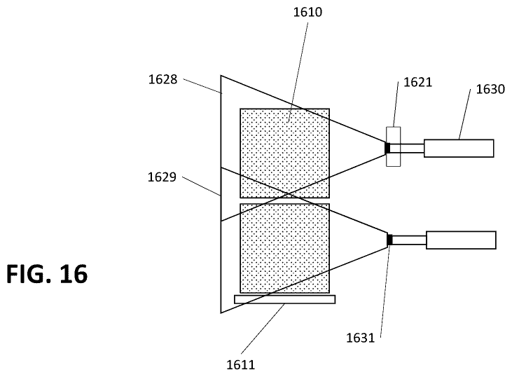

FIG. 16 schematically depicts the ultra-compact irradiation apparatus in a geometry/configuration suitable for phytosanitary and sterilization treatment in accordance with FIGS. 15A, 15B and 16C wherein one or more compact linac systems 1630 are stacked vertically alongside objects 1610 for in-line exposure to irradiate larger objects, such as stacks on pallet trays 1611. Directed radiation beams 1629 from emission target assemblies 1631 that may employ beam deflectors 1621 can produce overlapping radiation beams 1629 that can levelize and balance dose non-uniformities.

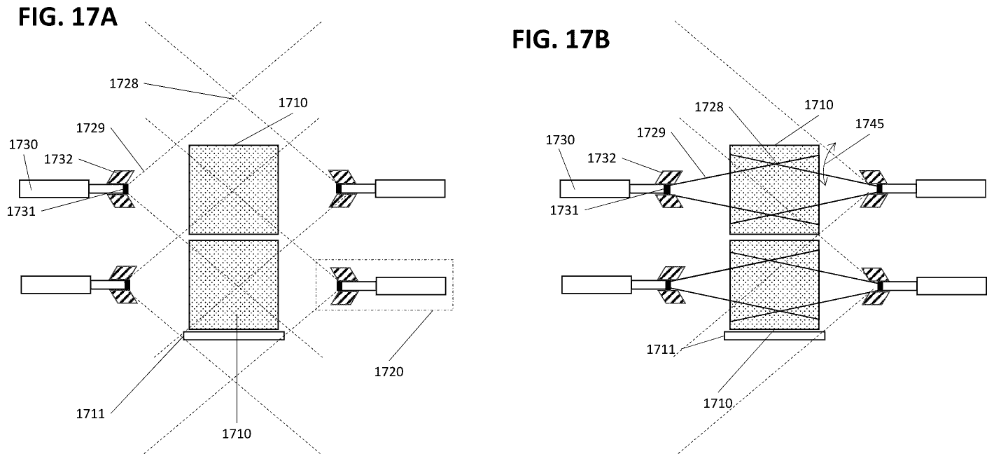

FIG. 17A and FIG. 17B schematically depict dynamic energy-angle 1745 control using relative position shield collimators 1748 to adjust a directed radiation beam (e.g. beam 1729) towards an object 1710 to be treated to adjust the relative dose profile across the object 1710 with overlapping radiation fields 1728 and control over the energy-angle distribution 1747. Each compact linac system 1730 can adjust its shield collimator 1732 to influence the dose profile.

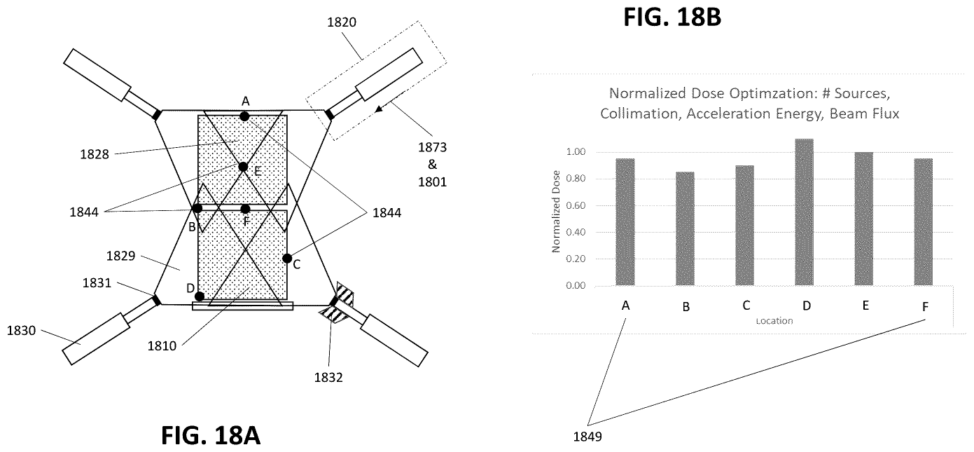

FIG. 18A and FIG. 18B schematically depict a feature of the present disclosure relating to placement of multiple—four in this example—compact linac systems 1820 in close proximity to objects 1810 positioned such that multiple directed radiation beams 1829 with overlapping radiation fields 1828 levelize a normalized dose (see position-dependent dose bar graph 1849) across objects 1810 to improve the dose uniformity ratio (DUR) and decrease the spread between Dₘₐₓ and Dₘᵢₙ. Dose control points 1844 correspond to columns in the graph for normalized dose 1849. By adjusting relative position 1848 of a shield collimator 1832 (z-cm), electron accelerator 1830 beam energy (MeV), beam current (mA) and duty cycle (% DF), and the presence of any low-energy shielding, and adjusting the longitudinal orientation 1873 and azimuthal placement 1801, the normalized doses depicted in bar graph 1849 can be adjusted.

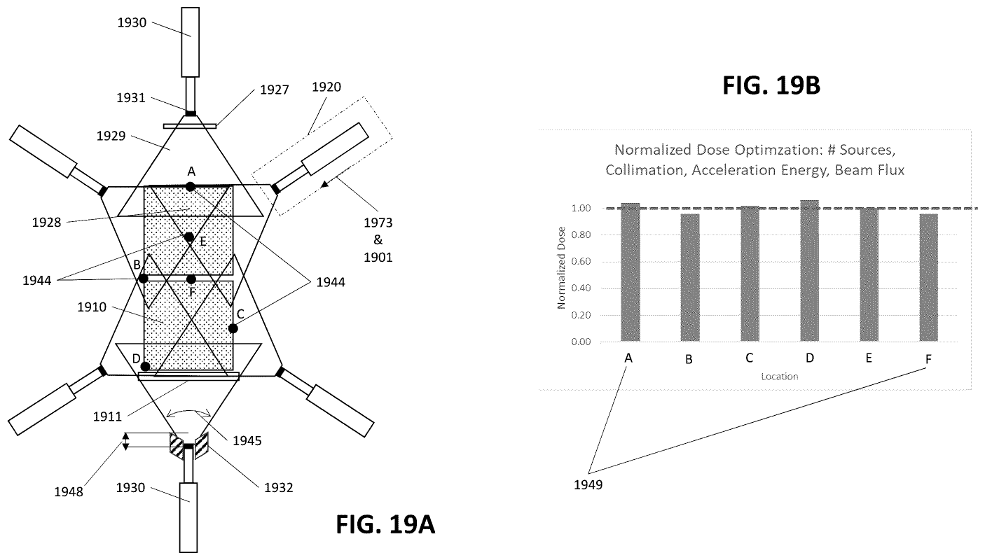

FIG. 19A and FIG. 19B further extend the schema of FIG. 18A and FIG. 18B by providing additional compact linac systems 1920 positioned around an object 1910 to be irradiated to further improve the dose uniformity ratio, decrease the spread between D max and Dₘᵢₙ, increase throughput and increase treatment speed. Also noted as in FIG. 18A-B, is the individual adjustment of each compact linac system 1920 “source”, adjustment of a shield collimator 1932 relative position 1948 for energy-angle control 1945, adjustment of acceleration energy, adjustment of low-energy filters 1927 for beam hardening, adjustment of beam current (flux), adjustment of beam position, adjustment of exposure time, rastering or beam deflection, adjusting the longitudinal orientation 1973 and azimuthal placement 1901, and other techniques described herein, can affect a normalized dose as shown in bar graph 1949. Dose control points 1944 correspond with normalized dose 1949 optimization across the object 1910. Each directed radiation beam 1929 from electron accelerators 1930 and emission target assemblies 1931 will generate overlapping radiation fields 1928 that can be tuned.

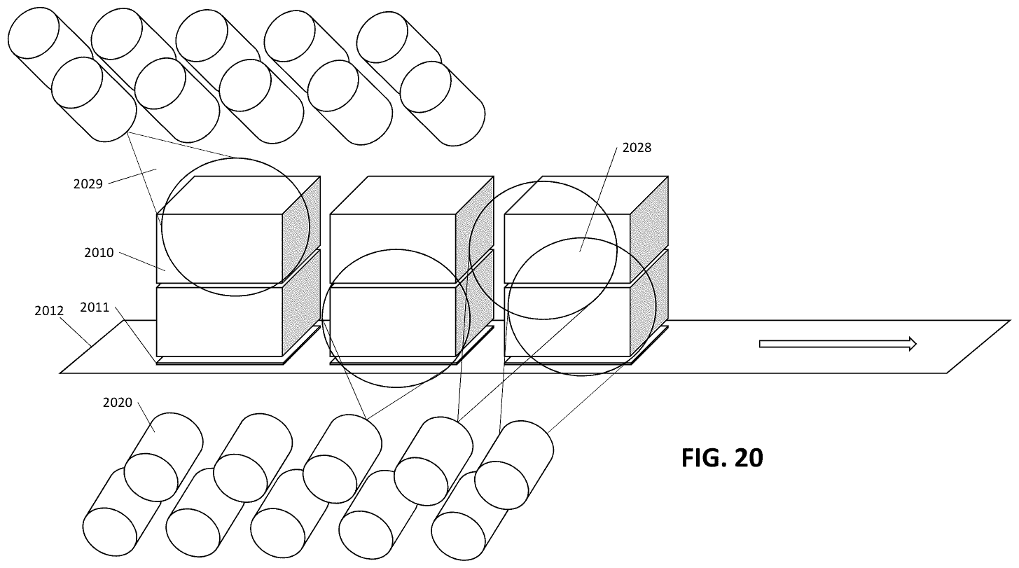

FIG. 20 schematically depicts an ultra-compact irradiation apparatus suitable for phytosanitary and sterilization treatment comprising many compact linac systems arrayed about an object conveyance for in-line exposure, e.g. 20-40, 4 kW units would project 80-160 kW beam power as a lower-cost, small facility size replacement for conventional large single-sided irradiation platforms. Here directed radiation beam 2029 from multiple compact linac systems 2020 produce overlapping radiation fields 2028 on object(s) 2010 stacked on pallet trays 2011 and moved on a conveyance 2012 apparatus. In this illustrative example, compact linac systems are configured as an array comprising multiple units positioned around the conveyance 2012 to an in-line, continuous, rapid ultra-compact irradiation system.

For example, a 160 kW high-power recirculating electron accelerator system may cost $5M in upfront capital expenditure for the machine plus $4M facility layout, conveyance and radiation shielding. A $0.1M 4 kW compact linac system that is multiplexed 40 times would cost $4M and have $2M cost for facility layout, conveyance and radiation shield due to the compact nature, need for 1 pass vs. 2-4 passes, and additional footprint and storage space. The capital expenditure savings of $3M is 50% of the ultra-compact irradiation apparatus $6M installation cost, and the system is scalable by adding units to scale throughput to encourage adoption.

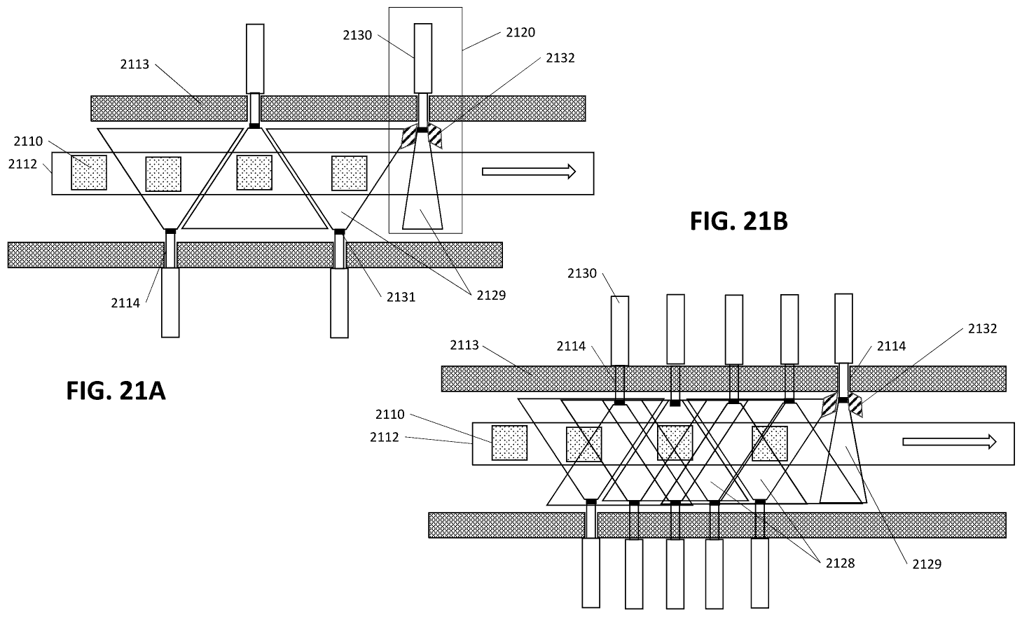

FIG. 21A and FIG. 21B schematically depict the ultra-compact irradiation apparatus employing local radiation shielding 2113 with small-diameter access ports 2114 for compact linac systems 2120 with emission target assemblies 2131 and shield collimators 2132 that greatly reduce backwards photons and stray radiation burden on facility, personnel and equipment; and enable close placement of compact linac systems 2120 to each other for overlapping radiation fields 2128, high area and volume density for dose uniformity and throughput of objects 2110 on conveyance 2112. The small-diameter emission target assemblies 2113 enable insertion through small-diameter access ports 2114 with shield collimator 2132 close coupling to provide excellent overall radiation shielding and containment within the object 2110 conveyance 2112 corridor. This lowers the total facility radiation shielding required.

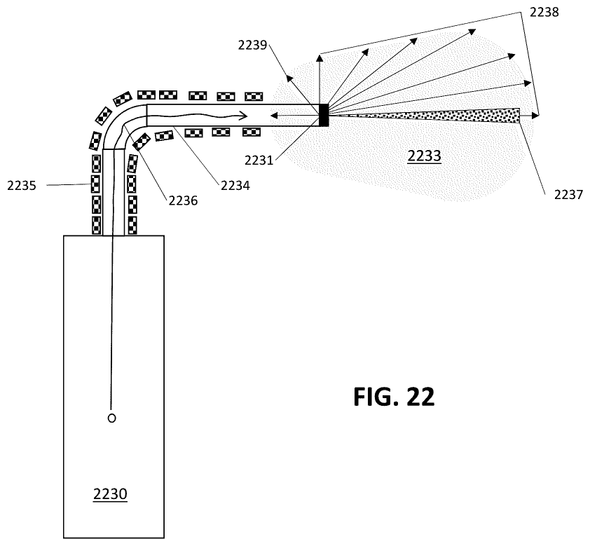

FIG. 22 schematically depicts another illustrative example in accordance with the present disclosure using magnetic electron transport to guide and direct the electron beam to an emission target assembly located further away from the electron accelerator or at an angle to the accelerator to produce a directed radiation beam. In this figure, a magnetic structure 2235 provides guidance for the electron trajectory 2236 along the non-linear drift tube 2234 towards the emission target assembly 2231 for produce emitted radiation 2233 in the form of a forward directed electron beam 2237 or forward photons 2238 and backward photons 2239.

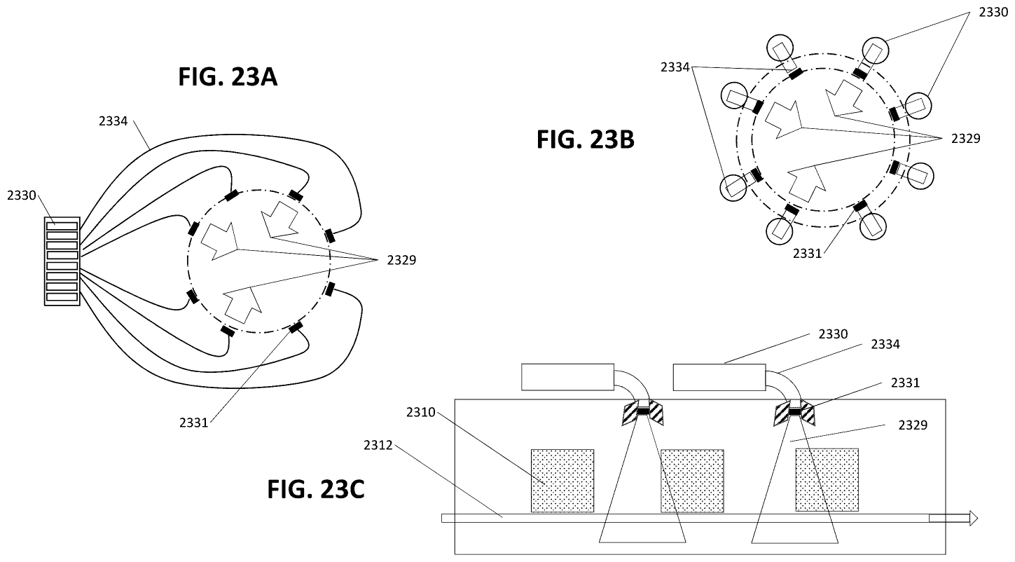

FIG. 23A further illustrates the feature introduced in FIG. 22 and is extended to multiple compact linac systems 2230 arranged to produce overlapping radiation fields from multiple directed radiation beams 2329 surrounding a region containing objects to be irradiated. By way of a particular example, FIG. 23B and FIG. 23C show the electron accelerators 2330 are positioned longitudinally along the conveyance axis 2312 and have one 90-degree turn for the electron beam transport drift tube 2334 into the emission target assembly 2331 firing radially inward towards the object 2310 at the center. Multiple compact linac systems can be arranged around the object for more uniform irradiation to approximate 360 degree illumination.

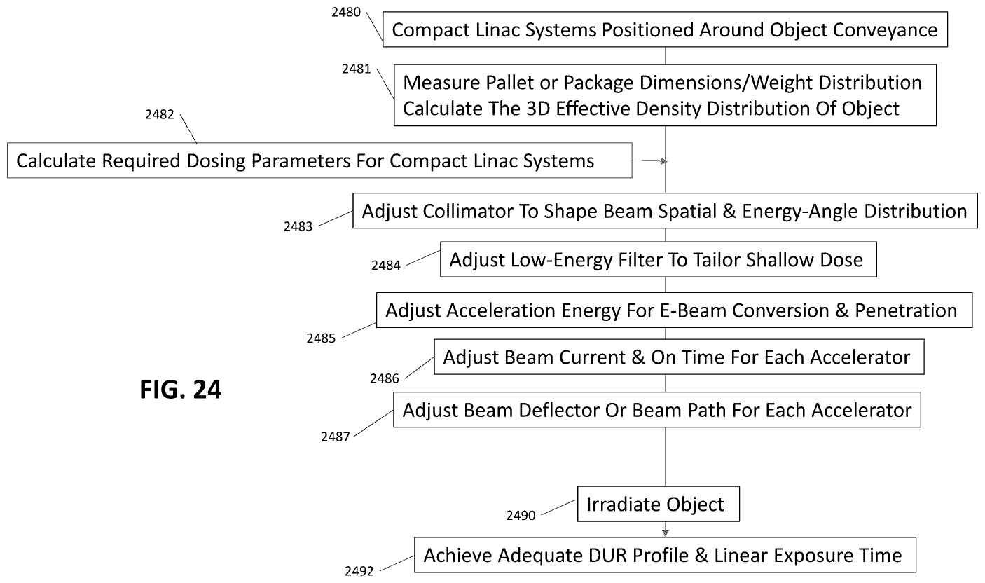

FIG. 24 a flowchart summarizes operations for a method that provides improving overall system efficiency for food irradiation, phytosanitary treatment, object exposure and sterilization. The method starts at 2480 with positioning compact linac systems around the object conveyance. Thereafter, during 2481 appropriate weights and volumes of the object are measured and used with other data inputs for determining an effective density distribution. This can be from multiple means, optical, mass sensors, distribution information, manifest information, attenuation scanning with x-rays, etc. The method further includes, during 2482, calculating the required dosing parameters for the compact linac systems for the object of interest using a targeted unit dose for treatment (determined by regulatory agency or customer need), the mass/density distribution on the volume of the object (obtained during 2481), position of compact linacs around the conveyance (from operation 2480) and desired conveyance speed. The method further includes calculating the required dosing strategy from 2482 for the given apparatus configuration with recommended adjustments to beam spatial and energy-angle distribution (operation 2483), energy filtering for beam hardening (operation 2484), acceleration energy and penetration (operation 2485), beam current and beam on time (fluence) (operation 2486), incorporation of a beam deflector or adjustment of beam path for rastering over the object (operation 2487) and the operation of each accelerator independently.

The method further includes adjusting (during 2483) shield collimators to shape beam distribution in space and energy; introducing or replacing a low-energy filter with different material and thickness for shallow dose attenuation (during 2484), adjustment of the acceleration energy over the course of the irradiation for photon peak spectrum profile or electron range vs. depth (during 2485); modulation of beam current (and duty factor %) for a given conveyor speed or dwell time (during 2486), and physical rastering or movement of the directed radiation beam over the object through beam deflection or overlapping radiation fields (during 2487). Thereafter, the ultra-compact irradiation apparatus is activated to irradiate the object 2490. The method achieves improved dose uniformity ratio and shorter linear exposure time started from an arrangement of compact linac systems positioned around the object conveyance 2492. Part of the calculation (operation 2482) is generating an effective density distribution over the 3D pallet/package/object volume with measurements and estimations to calculate the required dosing strategy for the given apparatus configuration. The disclosed system, comprising a plurality of compact linac systems, combined with the method described herein, enables making adjustments to the operation of each accelerator independently for independent beam spatial and energy-angle distribution, energy filtering for beam hardening, acceleration energy, beam current, and overlapping radiation fields.

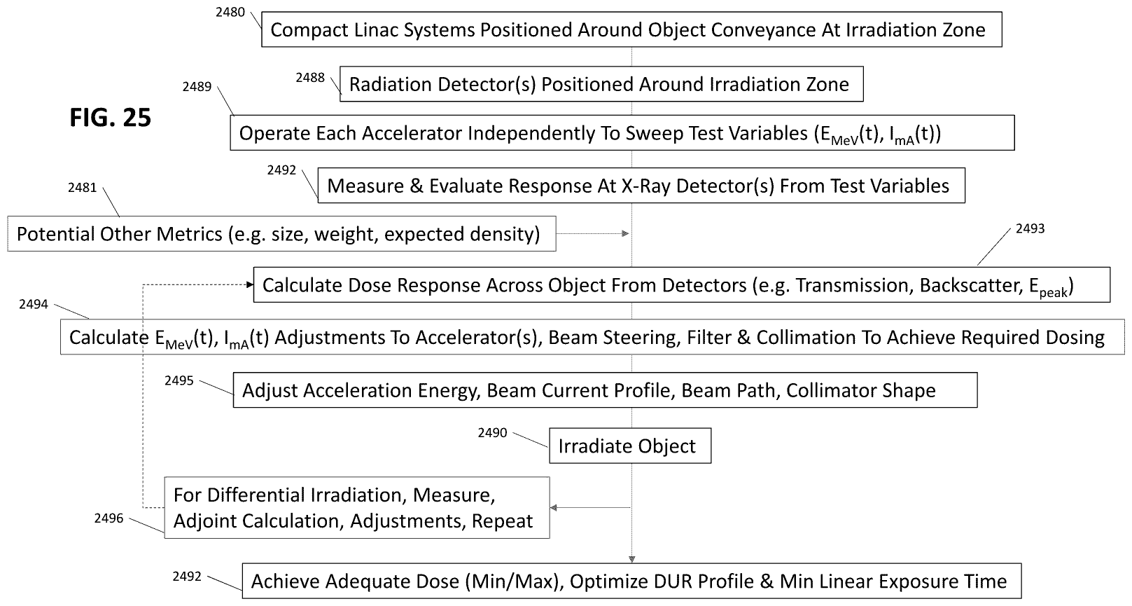

Expanding on the method of FIG. 24, the irradiation apparatus can incorporate in-situ feedback mechanisms to enable to determination of 2D areal and 3D volumetric dose profiles to the object under irradiation for quality control, dose mapping and certification. A challenge in phytosanitary irradiation is dose verification and traceable documentation for dose validation. The method described herein would complement the external NIST-tracible dosimetry tag and provide additional value-added quality assurance for food security users. FIG. 25 is a block diagram of another embodiment of the present disclosure detailing a method to use radiation detectors placed near the object to be irradiated to measure and evaluate the transmission, scattered and backscattered photon response from compact linac system operation with reference calibration sources and the actual object to be irradiated to calculate an effective dose response across the object, enable calculation of differential adjustments to the operational parameters (e.g. E_{MeV}(t), I_{mA}(t), accelerator beam time, beam steering, beam spatial and energy-angle adjustment, low-energy x-ray filter, collimation changes, etc.), and continue exposure to achieve the adequate dose, DUR profile, and exposure time. Operating each compact linac system independently enables differential object response functions and adjoint flux calculations for 3D dose mapping over the object. Using this differential irradiation response from multiple compact linac systems, an effective dosing strategy can be devised with parametric adjustment of accelerator conditions to achieve minimum dose threshold, not exceed dose ceiling, achieve optimized DUR and minimize exposure time.

The method starts with compact linac systems positioned around the object conveyance at the irradiation zone (operation 2480) and radiation detectors are positioned around the irradiation zone (operation 2488) with specific collimation and acceptance to define volumetric scan regions to collect transmitted and scanned radiations. Each compact linac system is operated independently to sweep test variables in terms of electron energy (MeV) and beam current (mA) (operation 2489) to generate directed radiation to interact, transmit through and scatter off the object and calibration reference sources. The method further comprises measuring and evaluating the response at the radiation detectors from the test variables from each compact linac system (operation 2492). Combined with other potential metrics, such as the expected 3D volumetric density distribution, during 2481, the method entails calculating the projected dose response from the test variables and other metrics based on the detected radiations during 2493. With this data a dose profile can be extrapolated over the 3D object to be irradiated for baseline operational parameter of the compact linac systems. The method further includes calculating required adjustments to the operational parameters from each compact linac system to achieve an adequate dose, DUR profile and exposure time (operation 2495). The method further describes making these adjustments to the operational parameters (also during operation 2495), e.g. acceleration energy, beam current, beam path, shield collimator position, beam on time, beam steering, low-energy filtering, duty factor %, etc. The object can be irradiated (during 2490) and a differential measurement can be taken during the irradiation to perform an adjoint calculation, make additional adjustments and repeat/continue irradiation (operation 2496) to further the goal of achieving adequate dose profiles over the object, defining min/max dose, optimizing DUR and/or minimizing linear exposure time for throughput and energy efficiency (operation 2492).

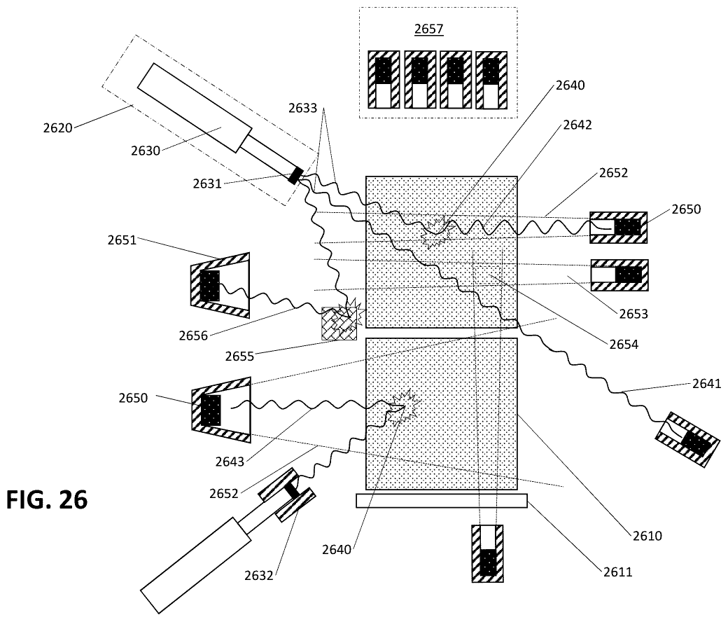

FIG. 26 is a detailed schematic illustration of an apparatus configured to achieve differential adjustment to radiation dosing and monitoring of the actual delivered dose conditions to the object under irradiation detailed in FIG. 25. In the illustrative example, compact linacs 2620 emit radiation 2633 that transmit on a path 2641 through an object 2610, have a scattering event 2640 and forward scatter 2652 through the object 2610 or have a scattering event 2640 and back scatter 2643 from the object 2610 or have a scattering event 2640 within a reference calibration source 2655 to produce calibration radiation 2656. The transmitted or scattered radiation is detected by a series of discrete detectors 2650, or arrays of detectors 2657, with suitable detector collimation 2651 provided/positioned around the object 2610 to be irradiated creating detector acceptance regions 2652 creating 2D dose integration volumes 2653 and 3D object voxel dose regions 2654. A differential object adjoint response function can be calculated for each compact linac contribution to the object irradiation for the method in FIG. 25 using superposition principles. Furthermore, the reference calibration sources 2655 also serve as a reference material in the directed radiation beams 2629 to provide known calibration checks for detector drift, gain and degradation over time without objects present in the conveyance and irradiation zone.

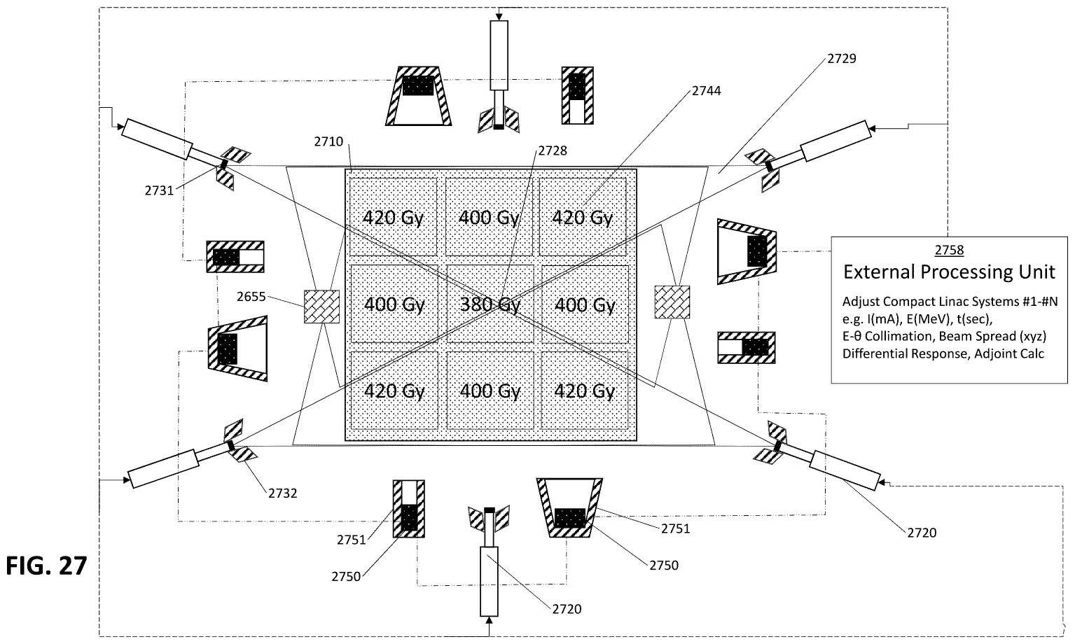

FIG. 27 is a schematic illustration of an apparatus configured to carry out the method of FIG. 25 and the apparatus of FIG. 26 for operational dose control (e.g. 400Gy±20Gy) and auditable verification for each object irradiated. Operating each compact linac system independently enables differential object response functions and adjoint flux calculations for 3D dose mapping over the object. A differential object adjoint response function can be calculated for each compact linac contribution to the object irradiation. Using the differential irradiation response from multiple compact linac systems, an effective dosing strategy can be devised with parametric adjustment of each accelerator conditions to sum and achieve minimum dose threshold, not exceed dose ceiling, achieve optimized DUR and minimize exposure time. Real-time feedback enables an external processing unit to calculate differential adjustments to the operational parameters (e.g. E_{MeV}(t), I_{mA}(t), accelerator beam time, beam steering, beam spatial and energy-angle adjustment, low-energy x-ray filter, collimation changes, etc.), and continue exposure to achieve the adequate dose, DUR profile, and exposure time.

In FIG. 27, directed beams of radiation 2729 are fired into an object 2710. Transmitted, scattered and calibration radiations are picked up by one or more detectors 2750 with detector collimations 2751 defining specific fields of view to generate 2D dose integration volumes and 3D object voxel dose regions. As each compact linac system 2720 is operated, the differential dose response can be measured and estimated from the adjoint calculation from the individual contributions to the detectors from the specific compact linac. An external processing unit 2758 rapidly calculates the differential responses from each compact linac and calculates an adjustment to the compact linac systems to optimize for a specific constraint: throughput, DUR max, min Gray dose, not to exceed maximum Gray dose, etc. The external processing unit 2758 sends commands to the compact linac systems 2720 to adjust parameters and execute the dose plan. The system can further measure the sum of dose response functions from all compact linacs to determine if there is any drift or additional correction needed. This method and apparatus can self-manage inconsistencies in the object product, say mixed fruits and vegetables, variations from box to box or batch to batch.

In summary, using 0.1-10 kW class sources allows for small-diameter emission target assemblies with closely-coupled shield collimators improving efficacy and lowering shield/facility costs. Multiple linac sources can be configured in an arrangement around the object to be irradiated to provide excellent dose uniformity and irradiation speed with tailored electron energy, collimation and beam current for precision dose control. Using multiple closely coupled electron accelerators and collimation for energy-angle selectivity, accelerator energy control and beam control, with low-energy shallow dose filtering it is possible to approach a DUR close to 1. In line conveyance for large pallets could be used for smaller facility footprint. Using small target converters on extended snouts on the electron accelerators, the very large scanning (rastering) horn used with conventional systems can be removed and the accelerators integrated with facility shielding that is compact and modular. Furthermore, using compact electron accelerators in the 0.1-10 kW class, multiple sources can be multiplexed around the object for scan-on-the-go without having to switch conveyors, rotate pallets or boxes of objects, and minimize start/stop and translation time loss, as well as reduce the overall footprint for conveyor systems vs. 2- and 4-point turnstiles with parallel pathways of costly conveyors and flipping mechanisms (for sealed boxes). Similarly, labor costs for unloading, unpacking, repacking and reloading can be mitigated.

It will be appreciated that the foregoing description relates to examples that illustrate a preferred configuration of the system. However, it is contemplated that other implementations of the invention may differ in detail from foregoing examples. As noted earlier, all references to the invention are intended to reference the particular example of the invention being discussed at that point and are not intended to imply any limitation as to the scope of the invention more generally. All language of distinction and disparagement with respect to certain features is intended to indicate a lack of preference for those features, but not to exclude such from the scope of the invention entirely unless otherwise indicated.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A food irradiation system comprising a plurality of compact linac systems,
wherein each compact linac system, of the plurality of compact linac systems, comprises:
a high energy particle beam source providing a particle beam at up to 10 MeV;
an emission target assembly configured to generate bremsstrahlung x-rays when impacted by particles of the particle beam; and
a drift tube through which the particle beam passes on a path from the high energy particle beam source to the emission target assembly,
wherein the emission target assembly is positioned at a distal end of the drift tube for direct impingement of the particle beam to generate the bremsstrahlung x-rays in a directed radiation beam, and
wherein ones of the plurality of compact linac systems are individually positioned such that, as a group, the plurality of compact linac systems provide directed radiation beam coverage at prescribed radiation dose levels for an overall cumulative volume.

2. The system of claim 1, wherein a shield collimator is provided external to the drift tube and proximal the emission target assembly, wherein the shield collimator defines a radiation dispersion pattern of the bremsstrahlung x-rays in the directed radiation beam.

3. The system of claim 2, wherein the shield collimator is configured for adjusting relative to the emission target assembly.

4. The system of claim 3, wherein the radiation dispersion pattern is modified spatially and energetically.

5. The system of claim 3, wherein the adjusting relative to the emission target assembly facilitates adjusting a radiation pattern of the bremsstrahlung x-rays in the directed radiation beam.

6. The system of claim 1 wherein the emission target assembly comprises a converter target material that, when impacted by particles of the particle beam, generates bremsstrahlung x-rays.

7. The system of claim 6, wherein the converter target material orientation is anisotropic.

8. The system of claim 1, wherein the high energy particle beam source is an electron accelerator.

9. The system of claim 1, wherein the emission target assembly permits passage of particles of the particle beam.

10. The system of claim 1 wherein the drift tube is configured as an extended snout.

11. The system of claim 1 wherein the path of the particle beam from the high energy particle beam source to the emission target assembly is a bending pathway defined by an external magnetic field source.

12. The system of claim 11, wherein the high energy beam sources of the plurality of compact linac systems, is maintained proximate other ones of the high energy beam sources, and wherein respective beams are guided on respective bending paths of respective drift tubes to individually positioned ones of the emission target assemblies oriented to provide the directed radiation beam coverage at prescribed radiation dose levels for an overall cumulative volume.

13. The system of claim 1 further comprising local radiation shielding positioned to contain energy of the directed radiation beam within an irradiation treatment space.

14. The system of claim 1 wherein at least one of the compact linac systems comprises a beam deflector positioned before the emission target assembly and configured to alter a trajectory of the particle beam towards a particular position on the emission target assembly.

15. The system of claim 1 further comprising at least one radiation detector positioned to receive energy from one or more of the compact linac systems, wherein the radiation detector provides information indicative of the directed radiation beam coverage at prescribed radiation dose levels for an overall cumulative volume.

16. The system of claim 15 further wherein the radiation detector is positioned within a detector collimator to define a volume from which the radiation detector receives radiation of the directed radiation beams of the compact linac systems.

17. The system of claim 16, further comprising a calibration unit positioned within the overall cumulative volume that produces calibration radiation sensed by the radiation detector.

18. A pallet irradiation system comprising:
a conveyance apparatus configured to carry a pallet supporting an object to be irradiated; and
the food irradiation system of claim 1.
